(19) United States
(12) Patent Application Publication
(10) Pub. No.: US 2025/0255180 A1
(43) Pub. Date: Aug. 7, 2025

(54) ORGANIC MATERIAL AND ORGANIC OPTOELECTRONIC DEVICE USING THE SAME

(71) Applicant: Raynergy Tek Incorporation, HSINCHU (TW)

(72) Inventor: YU-TANG HSIAO, HSINCHU (TW)

(21) Appl. No.: 19/035,384

(22) Filed: Jan. 23, 2025

Related U.S. Application Data
(60) Provisional application No. 63/550,636, filed on Feb. 7, 2024.

(51) Int. Cl. H10K 85/60; C07D 495/22; C07D 513/22; C07D 517/22; H10F 30/222; H10K 85/30

(52) U.S. Cl. CPC H10K 85/657; C07D 495/22; C07D 513/22; C07D 517/22; H10K 85/30; H10K 85/626; H10K 85/6576; H10F 30/222

(57) ABSTRACT

An organic material comprises a structure such as Formula I:

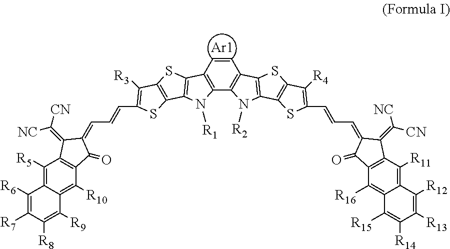

The structure contains polycyclic fused rings, diene groups, and naphthalene rings, which can effectively enhance the thermal stability of the material. The invention also provides an organic optoelectronic device comprising a first electrode, an active layer, and a second electrode. The active layer comprises the aforementioned organic material. This organic optoelectronic device exhibits great dark current density, detectivity, and photoelectric conversion efficiency in the near-infrared wavelength range, as well as superior thermal stability.

The present application is based on, and claims priority from, America provisional patent application number U.S. 63/550,636, filed on 2024 Feb. 7, and the disclosure of which is hereby incorporated by reference herein in its entirety.

### BACKGROUND OF THE INVENTION

### Field of the Invention

The present invention relates to an organic material applied in an organic optoelectronic device, and in particular to an organic optoelectronic device comprising the same.

### Description of the Prior Art

Compared to traditional inorganic optoelectronic devices, organic optoelectronic devices have wide absorption wavelength range, high absorption coefficient, and adjustable structures, and their light absorption range, energy level and solubility can be adjusted according to the target requirements. In addition, organic materials have the advantages of low cost, flexibility, low toxicity and large-area production of devices, so that organic optoelectronic devices have good competitiveness in various fields, such as organic field effect transistors (OFETs), organic light emitting diodes (OLEDs), organic photovoltaics (OPVs) and organic photodetectors (OPDs).

Organic photodetectors are semiconductor devices that convert optical signals into electrical signals. Within their operational wavelength range, the working conditions of organic photodetectors should feature high sensitivity, high response rate, minimal noise, small sensing area, low operating voltage, and high reliability. Organic photovoltaic cells, used for generating electricity from sunlight, have some similarities with organic photodetectors. The main difference between them is the purpose of use. The purpose of organic photovoltaics is to generate electricity, while organic photodetectors are devices that sense light and generate electronic signals. The light source for organic photovoltaic cells is sunlight, and their design primarily focuses on photoelectric conversion efficiency. In contrast, the light source for organic photodetectors is a specific wavelength emitter, whose intensity is much weaker than sunlight, which also makes the output electronic signal intensity lower. Therefore, the main consideration for organic photodetectors is detectivity. The factors affecting detectivity include responsivity and dark current density, which differ from those of organic photovoltaic cells. Another critical aspect of organic photodetectors is wavelength selection, which is different from organic photovoltaic cells. To avoid interference, organic photodetectors typically select infrared light. The main absorption range of the materials currently published in the literature is up to approximately 900 nm, with an optical bandgap of about 1.3 eV. The use of halogenated solvents in device manufacturing processes is less environmentally friendly.

Active layer materials play an important role in organic photodetectors and will directly affect device performance. The active layer material is divided into two parts: donor materials and acceptor materials. For the donor materials, the development of D-A conjugated polymers is the mainstream. The electron push-pull effect of electron-rich units and electron-deficient units in conjugated polymers can be used to control the energy levels and energy band gaps of polymers. The acceptor materials blended with the donor materials are usually fullerene derivatives with high conductivity, and its light absorption range is about 400-600 nm. However, the structure of fullerene derivatives is not easy to adjust, and their light absorption and energy levels are limited within a certain range, which limits the overall combination of the donor materials and the acceptor materials. With the development of the market, the demand for materials in the near-infrared region is gradually increasing. Even if the light absorption range of the conjugated polymer of the donor materials can be adjusted to the near-infrared region, it may not be able to have a good match due to the limitation of fullerene acceptor materials. Therefore, it is very important to develop non-fullerene acceptor materials to replace traditional fullerene acceptor materials in the breakthrough of active layer materials. As for the development of non-fullerene acceptor materials, in 2019, the ladder type molecules published by Yang et. al with the A-D-A′-D-A structure, such as Y6, have the light absorption range that can extend to the near-infrared region. However, the minimum optical band gap of the materials in the above mentioned literature is only about 1.3 eV, which is still insufficient for the applications with optical band gaps below 1.3 eV.

In the commercialization process of organic semiconductors, thermal stability is a critical factor. The temperature process includes the high temperatures required during device fabrication and the operating temperature of the device. In current technologies, organic photovoltaic cells generally do not require high-temperature processing during fabrication, so only the operating temperature needs to be considered. According to the literature, operating temperatures during summer are typically in the range of 50-80° C., with extreme cases potentially exceeding 100° C. Therefore, the thermal stability discussed in the literature is generally below 120° C. However, organic photodetectors differ from organic photovoltaic cells. The device manufacturing process requires the integration of semiconductor processes such as integrated circuits and color filters. The temperature of such processes is usually higher than 120° C. Therefore, in the process of device development, the thermal stability of organic photodetectors at high temperatures becomes more important.

As mentioned above, developing an organic material with an absorption spectrum extending into the near-infrared region, an optical bandgap lower than 1.25 eV, and compatibility with non-halogenated solvents for device fabrication, while achieving thermal stability above 120° C. for application in organic photodetectors, is a very important issue at present.

### SUMMARY OF THE INVENTION

In view of this, one category of the present invention is to provide an organic material comprises a structure such as Formula I:

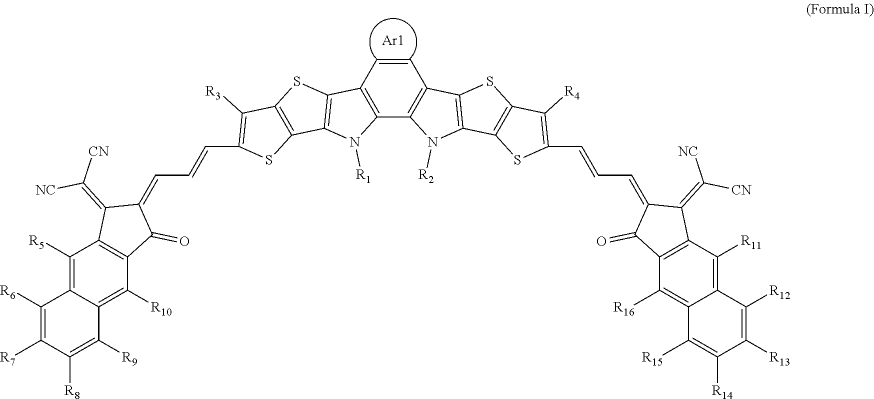

Wherein, Ar1 is a monocyclic ring or polycyclic ring comprising at least one five-membered heterocycle or six-membered heterocycle with one or more heteroatoms, and the heteroatoms are independently selected from at least one of S, N, O, and Se. R₁, R₂, R₃, and R₄ are independently selected from the following groups and their derivatives: C1-C30 alkyl, C1-C30 silyl, C1-C30 alkoxy, C1-C30 alkylthio, C1-C30 haloalkyl, C2-C30 ester, C1-C30 alkylaryl, C1-C30 alkyl heteroaryl, C1-C30 silyl aryl, C1-C30 silyl heteroaryl, C1-C30 alkoxyaryl, C1-C30 alkoxy heteroaryl, C1-C30 alkylthioaryl, C1-C30 alkylthio heteroaryl, C1-C30 haloalkylaryl, C1-C30 haloalkyl heteroaryl, C2-C30 esteryl aryl, and C2-C30 esteryl heteroaryl. R₅, R₆, R₇, R₈, R₉, R₁₀, R₁₁, R₁₂, R₁₃, R₁₄, R₁₅ and R₁₆ are independently selected from the following groups and their derivatives: C1-C30 alkyl, C1-C30 silyl, C1-C30 alkoxy, C1-C30 alkylthio, C1-C30 haloalkyl, halogen, hydrogen, deuterium, tritium, and cyano group.

Wherein, an optical band gap of the organic material is <1.25 eV.

Wherein, Ar1 is selected from the following structures:

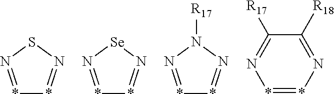

Wherein, each structure is connected by *, and R₁₇ and R₁₈ are independent single groups or connected to each other by covalent bonds to form a combined group.

Wherein, R₁₇ and R₁₈ are selected from the following groups and their derivatives: halogen, hydrogen, cyano group, C1-C30 alkyl, C2-C30 alkenyl, C1-C30 alkoxy, C1-C30 alkylthio, C1-C30 haloalkyl, C2-C30 ester, C1-C30 alkylaryl, C1-C30 alkyl heteroaryl, C1-C30 silyl aryl, C1-C30 silyl heteroaryl, C1-C30 alkoxyaryl, C1-C30 alkyloxy heteroaryl, C1-C30 alkylthioaryl, C1-C30 alkylthio heteroaryl, C1-C30 haloalkyl aryl, C1-C30 haloalkyl heteroaryl, C2-C30 esteryl aryl and C2-C30 esteryl heteroaryl.

Wherein, R₁ and R₂ are further independently selected from the following groups and their derivatives: C1-C30 alkyl, C1-C30 alkylaryl and C1-C30 alkyl heteroaryl; and R₃ and R₄ are further independently selected from the following groups and their derivatives: C1-C30 alkyl, C1-C30 silyl, C1-C30 alkoxy, C1-C30 alkylthio, C1-C30 alkylaryl, C1-C30 alkyl heteroaryl, C1-C30 alkoxyaryl, C1-C30 alkoxy heteroaryl, C1-C30 alkylthioaryl, C1-C30 alkylthio heteroaryl, C1-C30 haloalkyl aryl and C1-C30 haloalkyl heteroaryl.

Wherein, R₅, R₆, R₇, R₈, R₉, R₁₀, R₁₁, R₁₂, R₁₃, R₁₄, R₁₅ and R₁₆ are independently selected from the following groups: halogen, hydrogen, deuterium, and cyano group, and R₅, R₆, R₇, R₈, R₉, R₁₀, R₁₁, R₁₂, R₁₃, R₁₄, R₁₅ and R₁₆ are not hydrogen atoms at same time.

Wherein, R₅, R₁₀, R₁₁ and R₁₆ are further selected from hydrogen; and R₆, R₇, R₈, R₉, R₁₂, R₁₃, R₁₄ and R₁₅ are further independently selected from the following groups and their derivatives: halogen, hydrogen, C1-C5 haloalkyl, C1-C5 alkoxy, and cyano group.

The second category of the present invention is to provide an organic composition comprising at least one P-type organic semiconductor material and at least one N-type organic semiconductor material. The P-type organic semiconductor material comprises at least one of organic conjugated polymer or organic conjugated small molecule. The N-type organic semiconductor material comprises at least one organic material aforementioned.

The third category of the present invention is to provide an organic optoelectronic device comprising a first electrode, an active layer and a second electrode. The active layer at least comprises an organic material aforementioned. Wherein, the active layer is disposed between the first electrode and the second electrode, and at least one of the first electrode and the second electrode is a transparent or semi-transparent electrode.

The fourth category of the present invention is to provide an organic optoelectronic device comprising a first electrode, an active layer and a second electrode. The active layer at least comprises an organic composition aforementioned. Wherein, the active layer is disposed between the first electrode and the second electrode, and at least one of the first electrode and the second electrode is a transparent or semi-transparent electrode.

Compared with the prior art, the organic material of the present invention has polycyclic condensed rings, diene groups and naphthalene rings, which its absorption spectrum extends to the near-infrared region, and the optical band gap is lower than 1.25 eV. Furthermore, the organic material of the present invention can be processed by using non-halogen solvents in device fabrication. The organic photodetectors comprising the organic material has good dark current density, detectivity, and photoelectric conversion efficiency in the near-infrared wavelength range, along with outstanding thermal stability.

### BRIEF DESCRIPTION OF THE APPENDED DRAWINGS

Some of the embodiments will be described in detail, with reference to the following figures, wherein like designations denote like members, wherein:

FIG. 1 shows a schematic structural diagram of one embodiment of an organic optoelectronic device of the present invention.

FIG. 2 shows absorption spectra in thin-film state of Comparative Example 1, Example 1, Example 2, Example 3, and Example 6 of the organic materials.

FIG. 3 shows the thin-film absorption spectra of Comparative Example 1 of the organic materials with and without annealing.

FIG. 4 shows the thin-film absorption spectra of Example 1 of the organic materials with and without annealing.

FIG. 5 shows the thin-film absorption spectra of Example 2 of the organic materials with and without annealing.

FIG. 6 shows the thin-film absorption spectra of Example 3 of the organic materials with and without annealing.

FIG. 7 shows the thin-film absorption spectra of Example 6 of the organic materials with and without annealing.

### DETAILED DESCRIPTION OF THE INVENTION

In order to make the advantages, spirit and features of the present invention easier and clearer, it will be detailed and discussed in the following with reference to the embodiments and the accompanying drawings. It is worth noting that the specific embodiments are merely representatives of the embodiments of the present invention, but it can be implemented in many different forms and is not limited to the embodiments described in this specification. Rather, these embodiments are provided so that this disclosure will be thorough and complete.

The terminology used in the various embodiments disclosed in the present invention is only for the purpose of describing specific embodiments, and is not intended to limit the various embodiments disclosed in the present invention. As used herein, singular forms also include plural forms unless the context clearly indicates otherwise. Unless otherwise defined, all terms (including technical and scientific terms) used in this specification have the same meanings as commonly understood by one of ordinary skill in the art to which the various embodiments disclosed herein belong. The above terms (such as those defined in commonly used dictionaries) will be interpreted as having the same meaning as the contextual meaning in the same technical field, and will not be interpreted as having an idealized or overly formal meaning, unless explicitly defined in the various embodiments disclosed herein.

In the description of this specification, the description of the reference terms “an embodiment”, “a specific embodiment” and the like means that specific features, structures, materials, or characteristics described in connection with the embodiment are included in at least one embodiment of the present invention. In this specification, the schematic expressions of the above terms do not necessarily refer to the same embodiment. Furthermore, the particular features, structures, materials, or characteristics described may be combined in any suitable manner in any one or more embodiments.

### Definition

As used herein, “donor” material and “p-type” (“P-type”) material refer to a semiconductor material, such as an organic semiconductor material, having holes as a primary current or charge carrier. In some embodiments, when a p-type semiconductor material is deposited on a substrate, it can provide the hole mobility greater than about 10⁻⁵ cm²/Vs.

As used herein, “acceptor” material and “n-type” (“N-type”) material refer to the semiconductor material, such as the organic semiconductor material, having electrons as the primary current or the charge carrier. In some embodiments, when an n-type semiconductor material is deposited on a substrate, it can provide the electron mobility of more than about 10⁻⁵ cm²/Vs.

“*” or “*” in the structures listed herein represents the available bonding positions of this structure, but not limited thereto.

As used herein, “solution process” refers to a process in which a compound (e.g., a polymer), material, or composition can be used in a solution state, such as spin coating, printing (e.g., inkjet printing, gravure printing, and lithography printing), spray coating, slit coating, drop casting, dip coating, and blade coating.

As used herein, “annealing” refers to a post-deposition thermal treatment to a semi-crystalline polymer film for certain duration in the environment or under decompressed or pressurized environment. “Annealing temperature” refers to the temperature at which the polymer film or the mixed film of the polymer and other molecules can perform small-scale molecular movement and rearrangement during the annealing process. Without being limited by any particular theory, it is believed that annealing can lead to an increase in crystallinity in the polymer film and enhance the carrier mobility of the polymer film or a mixed film formed by the polymer and other molecules, and the molecules are arranged alternately to achieve the effect of independent transporting paths of effective electrons and holes.

The external quantum efficiency (EQE) as used herein is the spectral response Amp/Watt unit, which Amp is converted to the number of electrons per unit time (electron/sec) and Watt is converted to the number of photons per unit time (Photons/sec), and insert the quantum efficiency obtained by the above formula. Generally speaking, quantum efficiency (QE) refers to external quantum efficiency (EQE), also known as incident photon-electron conversion efficiency (IPCE).

Dark current density (J_{d} or J_{dark}) as used herein, also known as no-illumination current, refers to the current flows in an optoelectronic device in the absence of light irradiation.

The responsibility (R) and the detectivity (D*) as used herein are based on measuring the dark current and external quantum efficiency (EQE) of the organic photodetector, and are calculated by the following formula:

$$R ( λ ) = EQE \frac{{λ}_{q}}{hc} , {D}^{*} = \frac{( λ / 1240 ) \times ( E Q E )}{\sqrt{2 e {J}_{d a r k}}}$$

Wherein, λ is the wavelength, e is the elementary charge (1.602×10⁻¹⁹ Coulombs), h is Planck's constant (6.626×10⁻³⁴ m² kg/s), c is the speed of light (3×10⁸ m/sec), and J_{dark} is the dark current.

In an embodiment, an organic material comprises a structure such as Formula I:

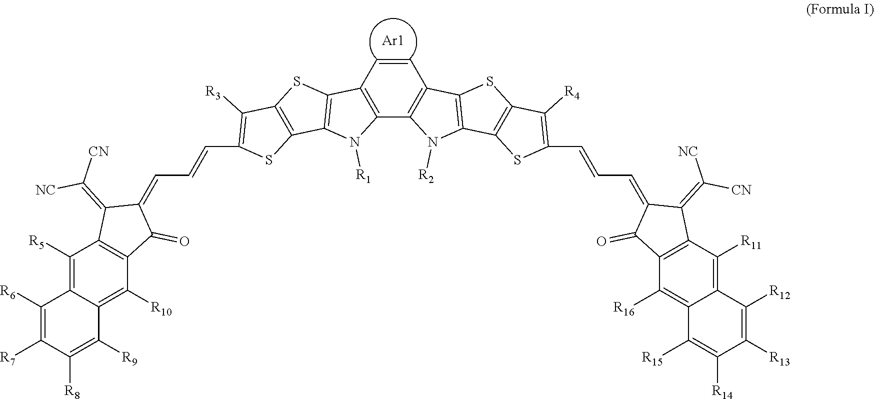

Wherein, Ar1 is a monocyclic ring or polycyclic ring comprising at least one five-membered heterocycle with or without substituents or six-membered heterocycle with or without substituents with one or more heteroatoms. The heteroatoms are independently selected from at least one of S, N, O, and Se. R₁, R₂, R₃, and R₄ are independently selected from the following groups: C1-C30 alkyl with or without substituents, C1-C30 silyl with or without substituents, C1-C30 alkoxy with or without substituents, C1-C30 alkylthio with or without substituents, C1-C30 haloalkyl with or without substituents, C2-C30 ester with or without substituents, C1-C30 alkylaryl with or without substituents, C1-C30 alkyl heteroaryl with or without substituents, C1-C30 silyl aryl with or without substituents, C1-C30 silyl heteroaryl with or without substituents, C1-C30 alkoxyaryl with or without substituents, C1-C30 alkoxy heteroaryl with or without substituents, C1-C30 alkylthioaryl with or without substituents, C1-C30 alkylthio heteroaryl with or without substituents, C1-C30 haloalkyl aryl with or without substituents, C1-C30 haloalkyl heteroaryl with or without substituents, C2-C30 ester aryl with or without substituents, and C2-C30 ester heteroaryl with or without substituents. R₅, R₆, R₇, R₈, R₉, R₁₀, R₁₁, R₁₂, R₁₃, R₁₄, R₁₅ and R₁₆ are independently selected from the following groups: C1-C30 alkyl with or without substituents, C1-C30 silyl with or without substituents, C1-C30 alkoxy with or without substituents, C1-C30 alkylthio with or without substituents, C1-C30 haloalkyl with or without substituents, halogen, hydrogen, deuterium, tritium, and cyano group. This structure has good optical properties and an appropriate energy gap, and can be used with suitable p-type materials to make organic optoelectronic devices. Wherein, this structure has polycyclic condensed rings, diene groups and naphthalene rings. The structure of the present invention includes the following characteristics: 1. the optical band gap is lower than 1.25 eV; and 2. it has good thermal stability.

In a preferred embodiment, Ar1 is selected from one of the following structures:

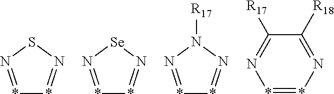

Wherein, each structure is connected by *, and R₁₇ and R₁₈ are independent single groups or connected to each other by covalent bonds to form a combined group. In practice, R₁₇ and R₁₈ are selected from the following groups: halogen, hydrogen, cyano, C1-C30 alkyl with or without substituents, C2-C30 alkenyl with or without substituents, C1-C30 alkoxy with or without substituents, C1-C30 alkylthio with or without substituents, C1-C30 haloalkyl with or without substituents, C2-C30 ester with or without substituents, C1-C30 alkylaryl with or without substituents, C1-C30 alkyl heteroaryl with or without substituents, C1-C30 silyl aryl with or without substituents, C1-C30 silyl heteroaryl with or without substituents, C1-C30 alkoxyaryl with or without substituents, C1-C30 alkoxy heteroaryl with or without substituents, C1-C30 alkylthioaryl with or without substituents, C1-C30 alkylthio heteroaryl with or without substituents, C1-C30 haloalkyl aryl with or without substituents, C1-C30 haloalkyl heteroaryl with or without substituents, C2-C30 esteryl aryl with or without substituents and C2-C30 esteryl heteroaryl with or without substituents.

In practice, R₁ and R₂ are further independently selected from the following groups: C1-C30 alkyl with or without substituents, C1-C30 alkylaryl with or without substituents and C1-C30 alkyl heteroaryl with or without substituents. R₃ and R₄ are further independently selected from the following groups: C1-C30 alkyl with or without substituents, C1-C30 silyl with or without substituents, C1-C30 alkoxy with or without substituents, C1-C30 alkylthio with or without substituents, C1-C30 alkylaryl with or without substituents, C1-C30 alkyl heteroaryl with or without substituents, C1-C30 alkoxyaryl with or without substituents, C1-C30 alkoxy heteroaryl with or without substituents, C1-C30 alkylthioaryl with or without substituents, C1-C30 alkylthio heteroaryl with or without substituents, C1-C30 haloalkyl aryl with or without substituents and C1-C30 haloalkyl heteroaryl with or without substituents.

In practice, R₅, R₆, R₇, R₈, R₉, R₁₀, R₁₁, R₁₂, R₁₃, R₁₄, R₁₅ and R₁₆ are independently selected from the following groups: halogen, hydrogen, deuterium, and cyano group, and R₅, R₆, R₇, R₈, R₉, R₁₀, R₁₁, R₁₂, R₁₃, R₁₄, R₁₅ and R₁₆ are not hydrogen atoms at same time. In a preferred embodiment, R₅, R₁₀, R₁₁ and R₁₆ are further selected from hydrogen. R₆, R₇, R₈, R₉, R₁₂, R₁₃, R₁₄ and R₁₅ are further independently selected from the following groups: halogen, hydrogen, C1-C5 haloalkyl with or without substituents, C1-C5 alkoxy with or without substituents, and cyano group.

In details, the organic material could comprise the following example 1 to example 22:

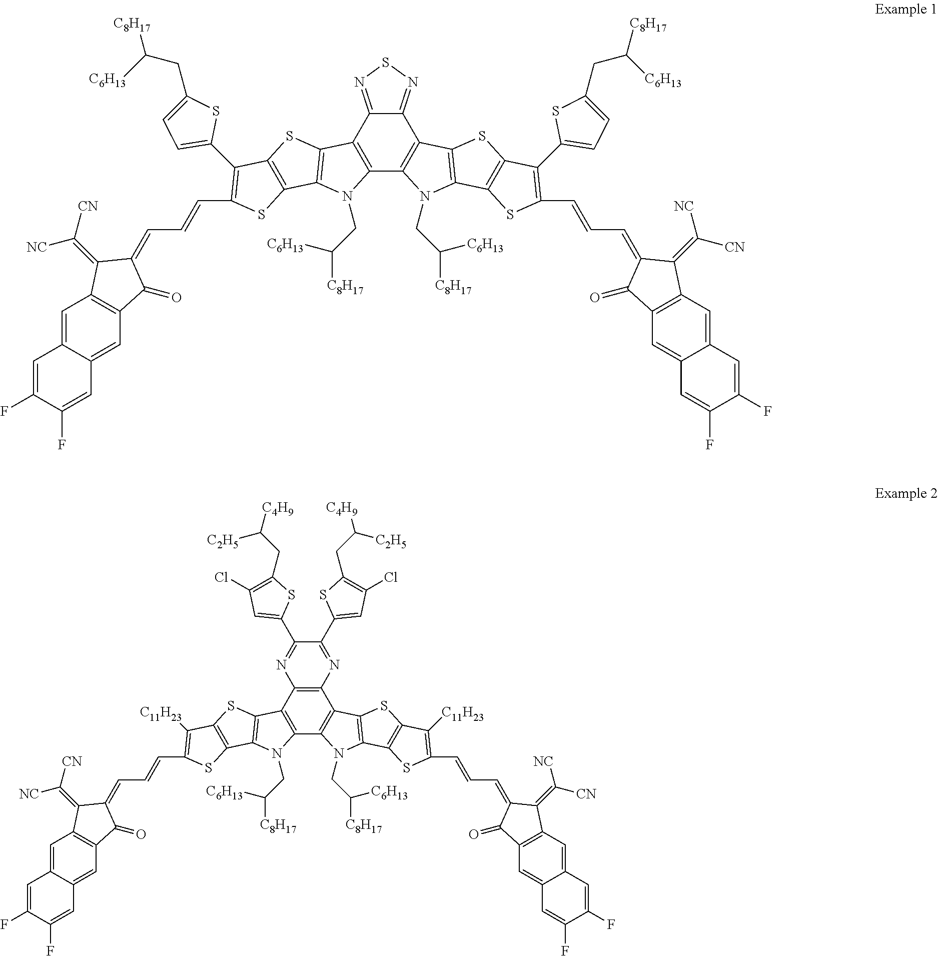

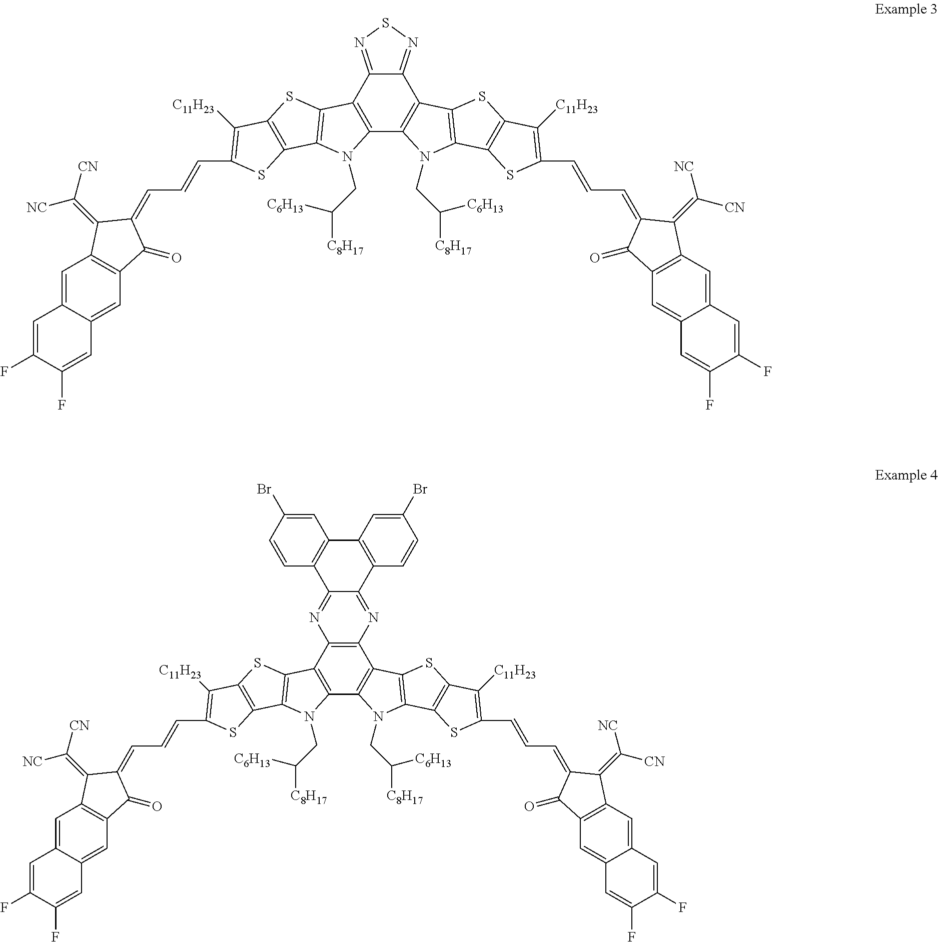

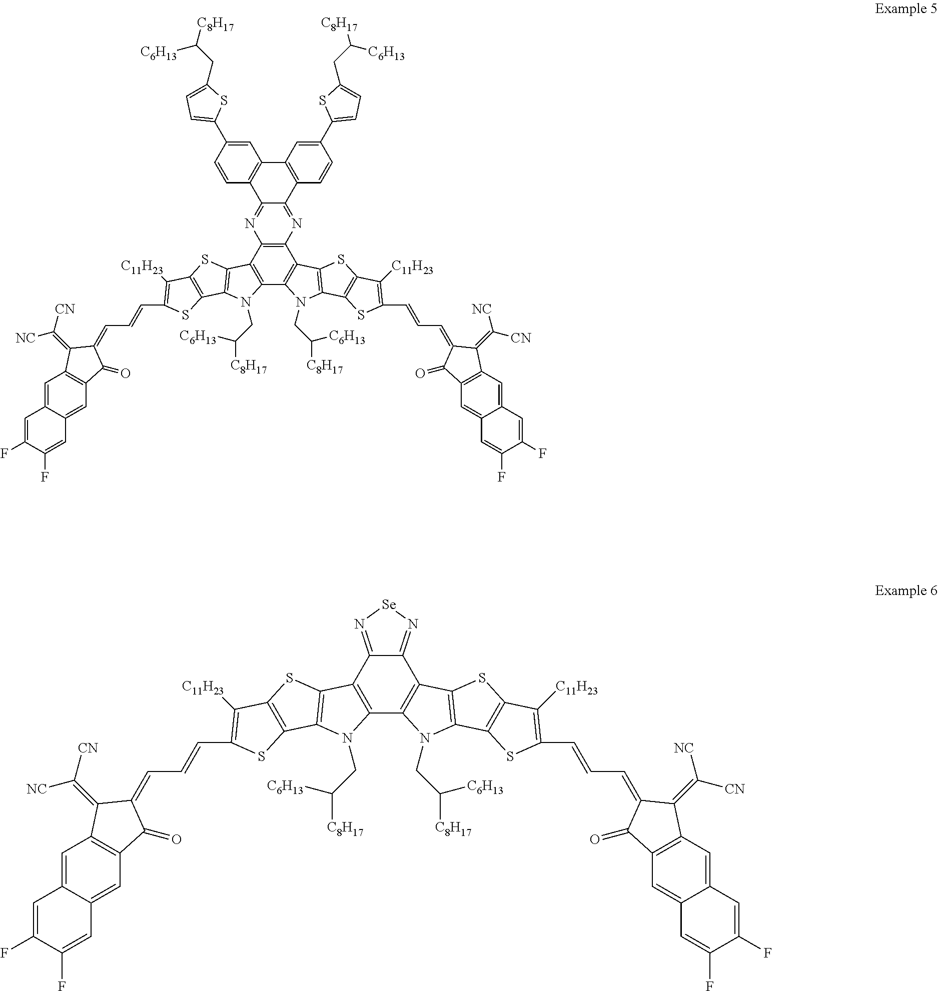

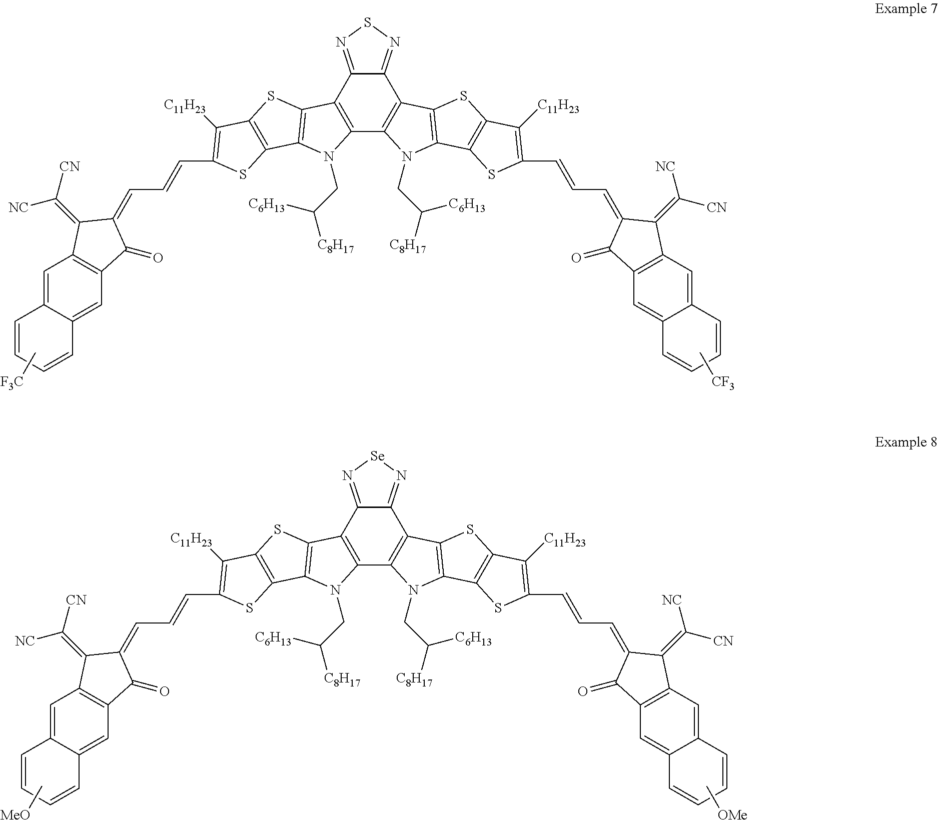

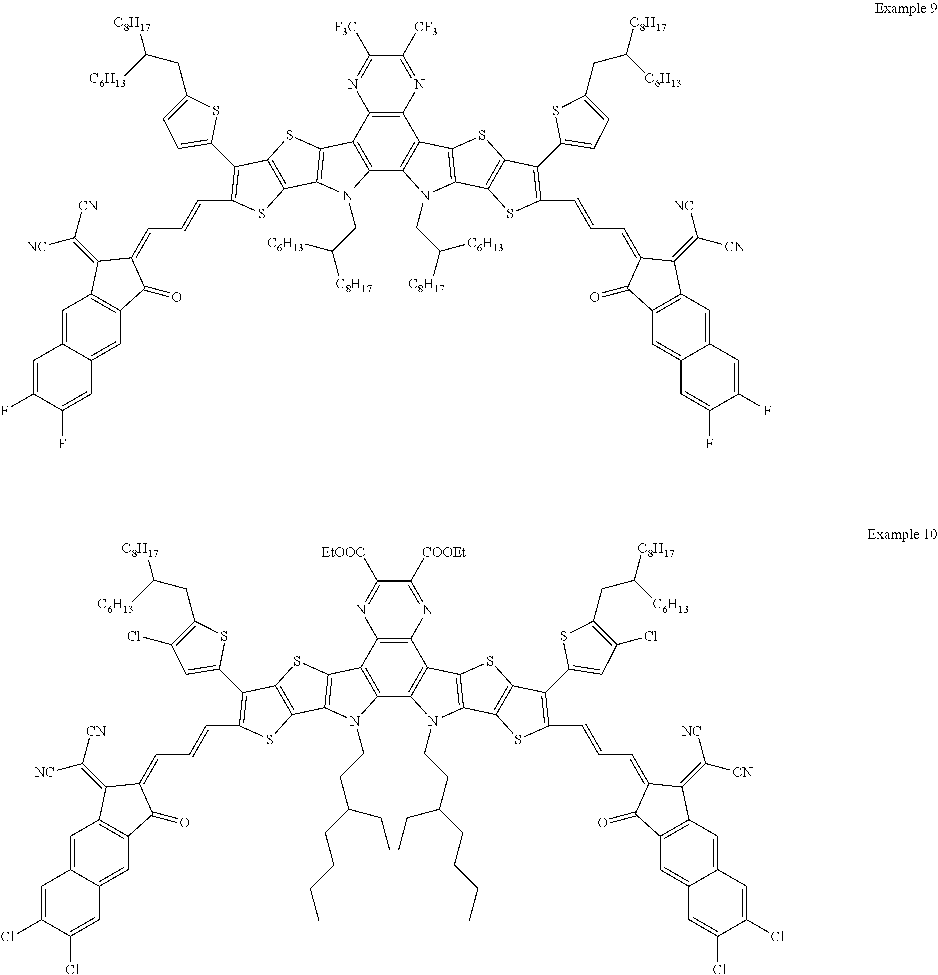

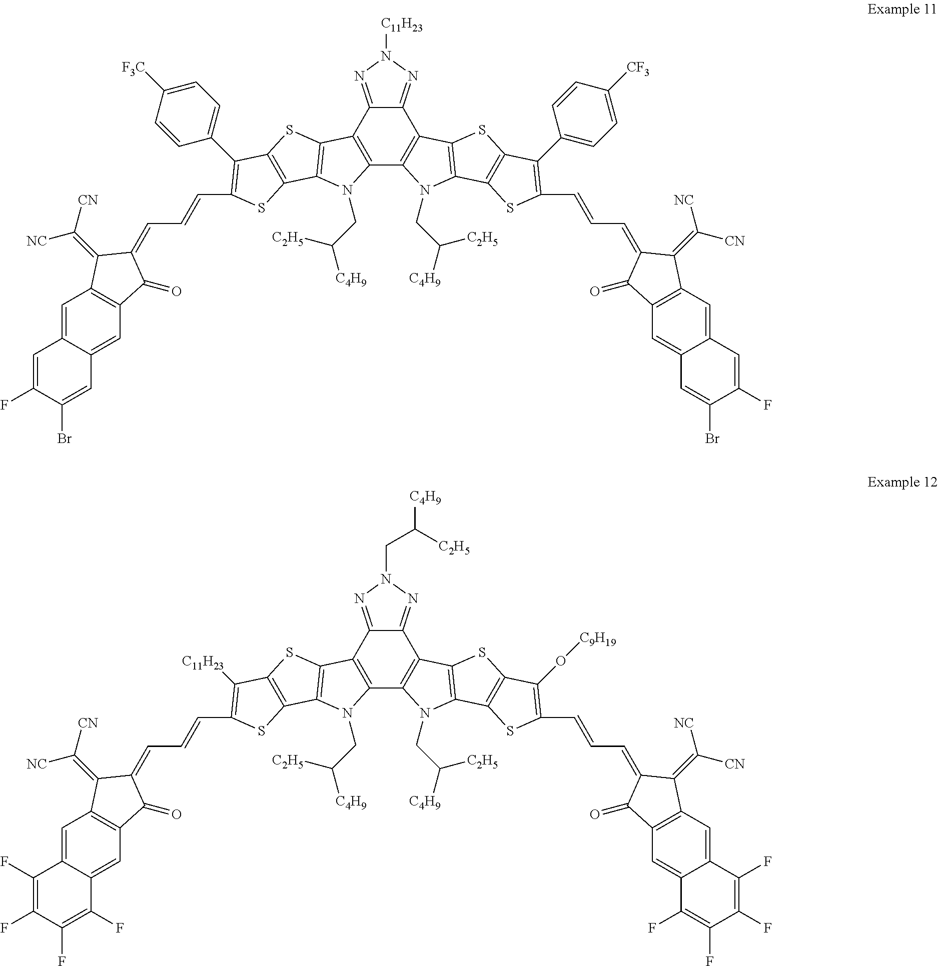

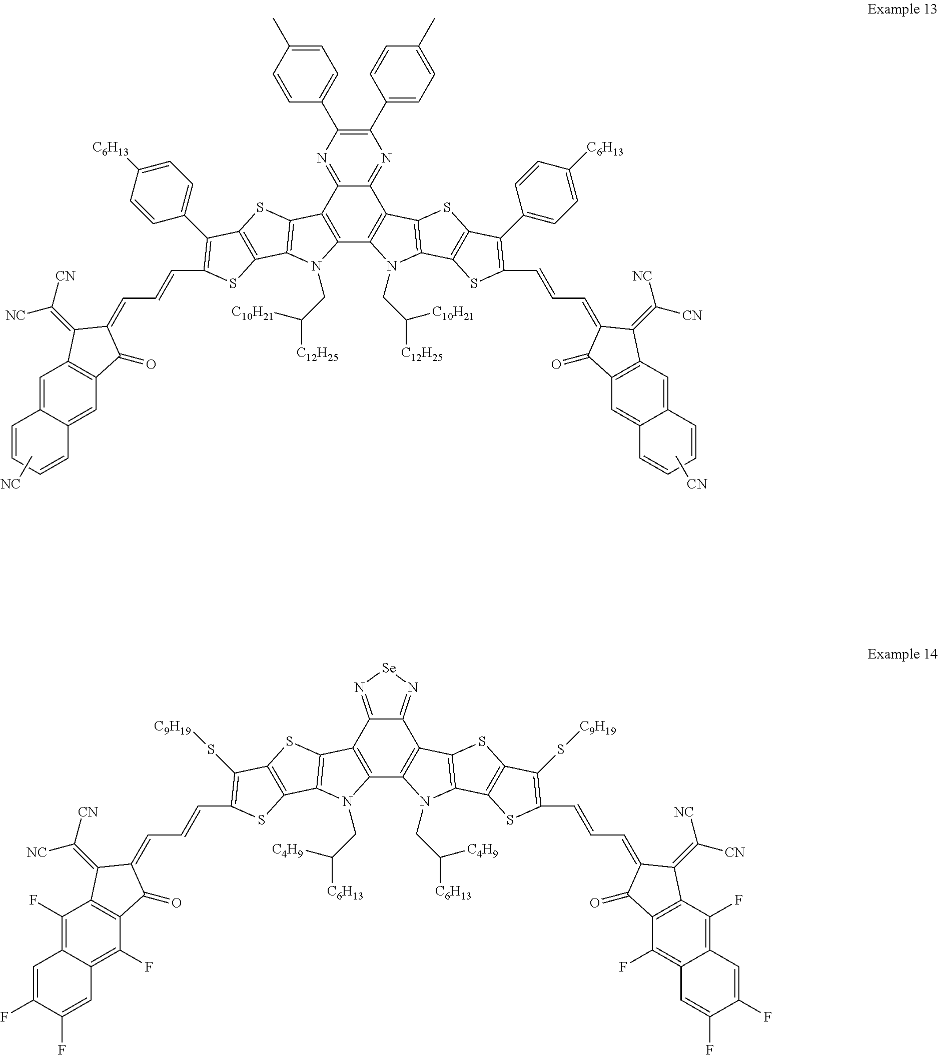

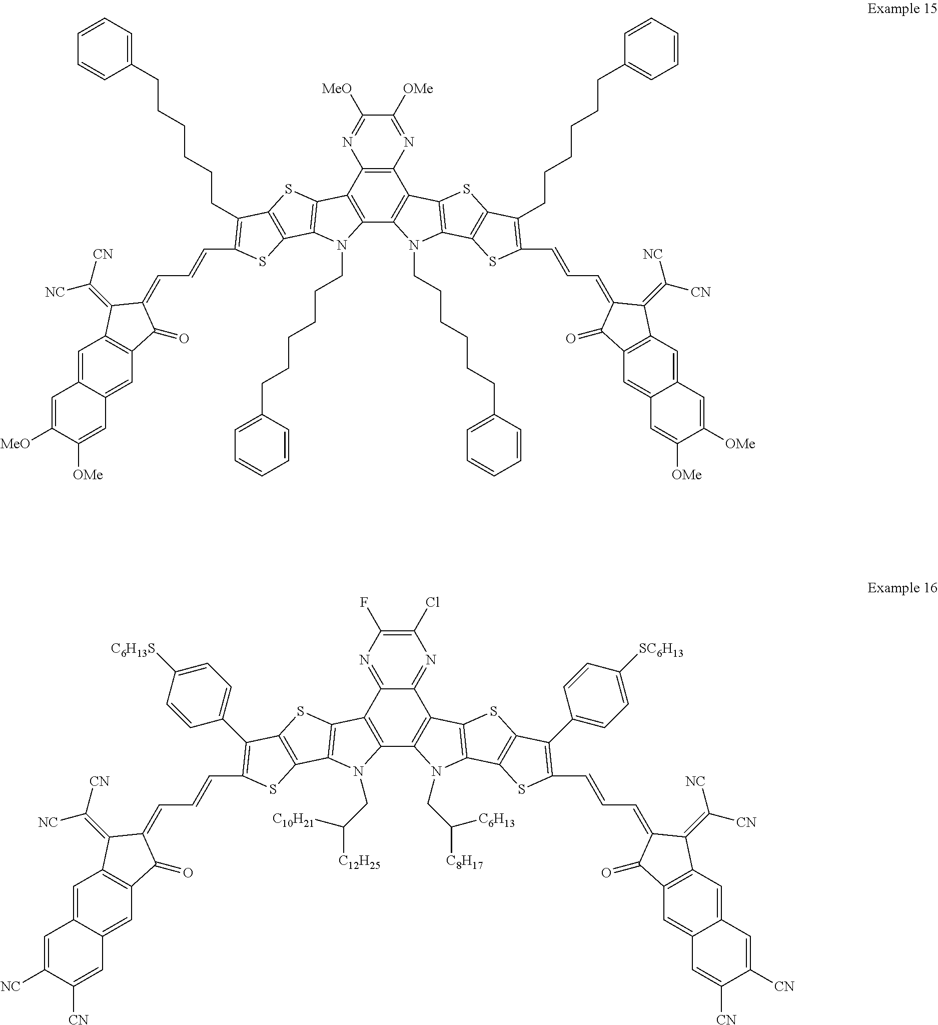

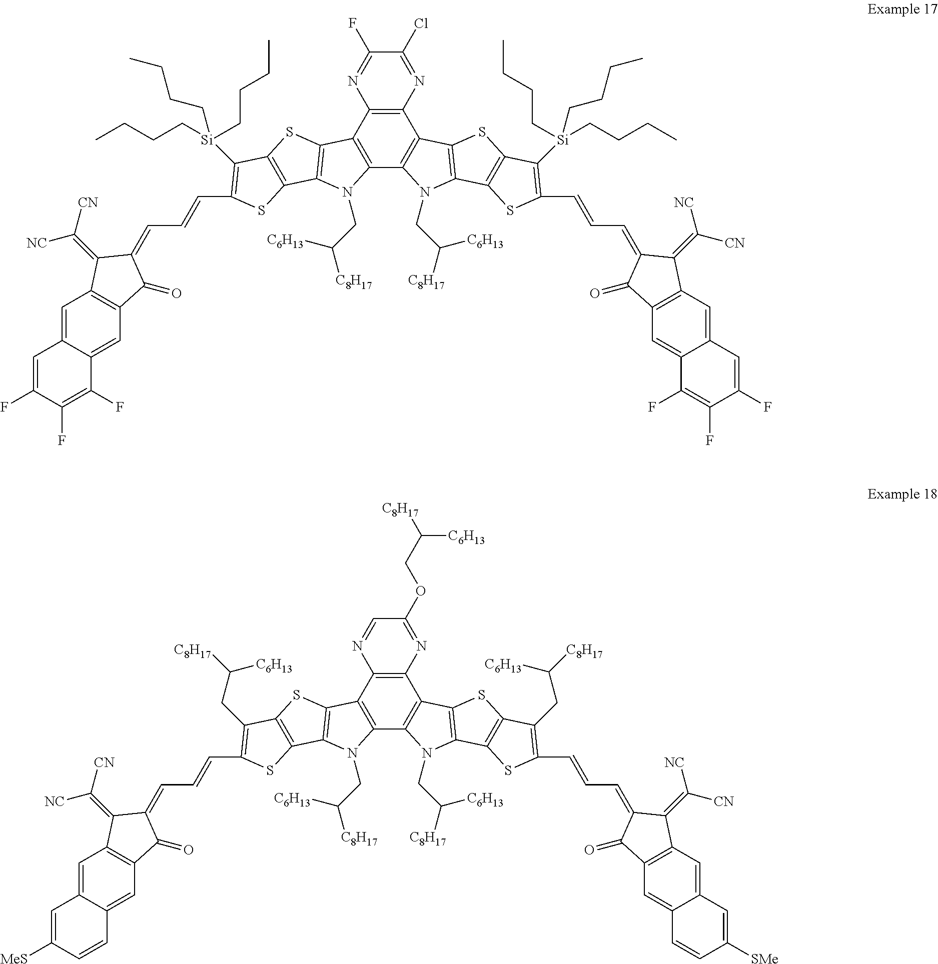

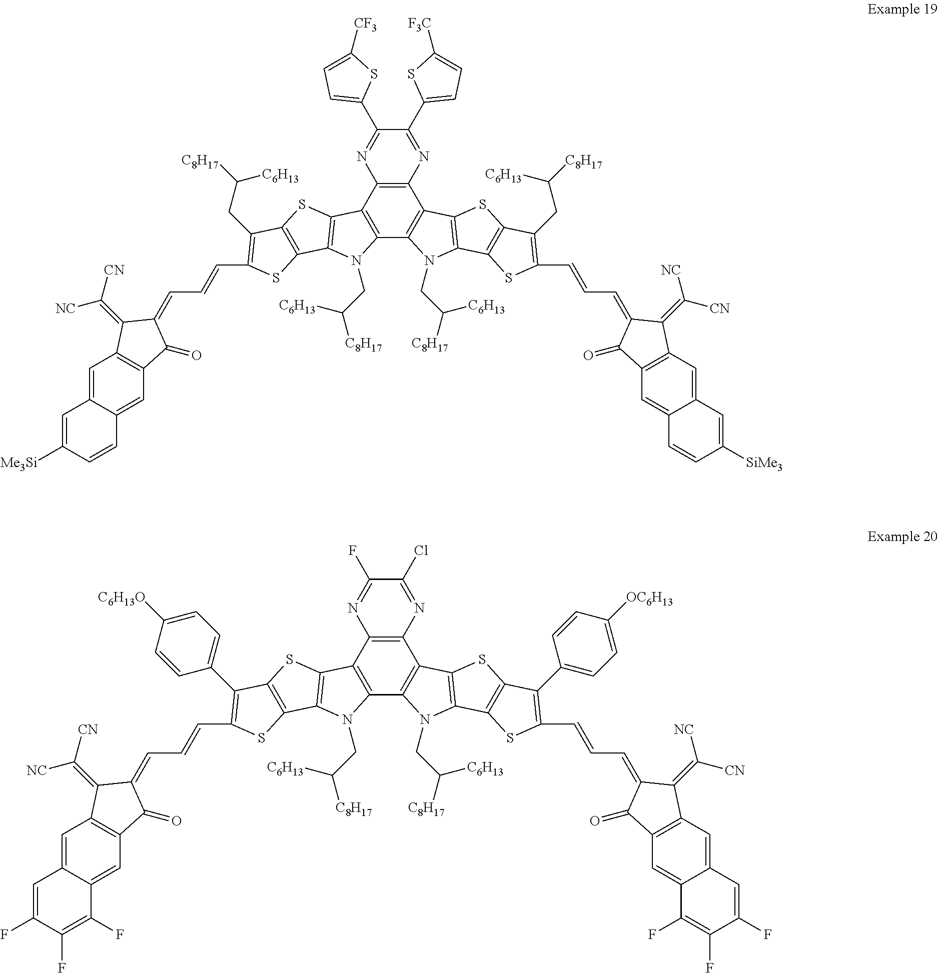

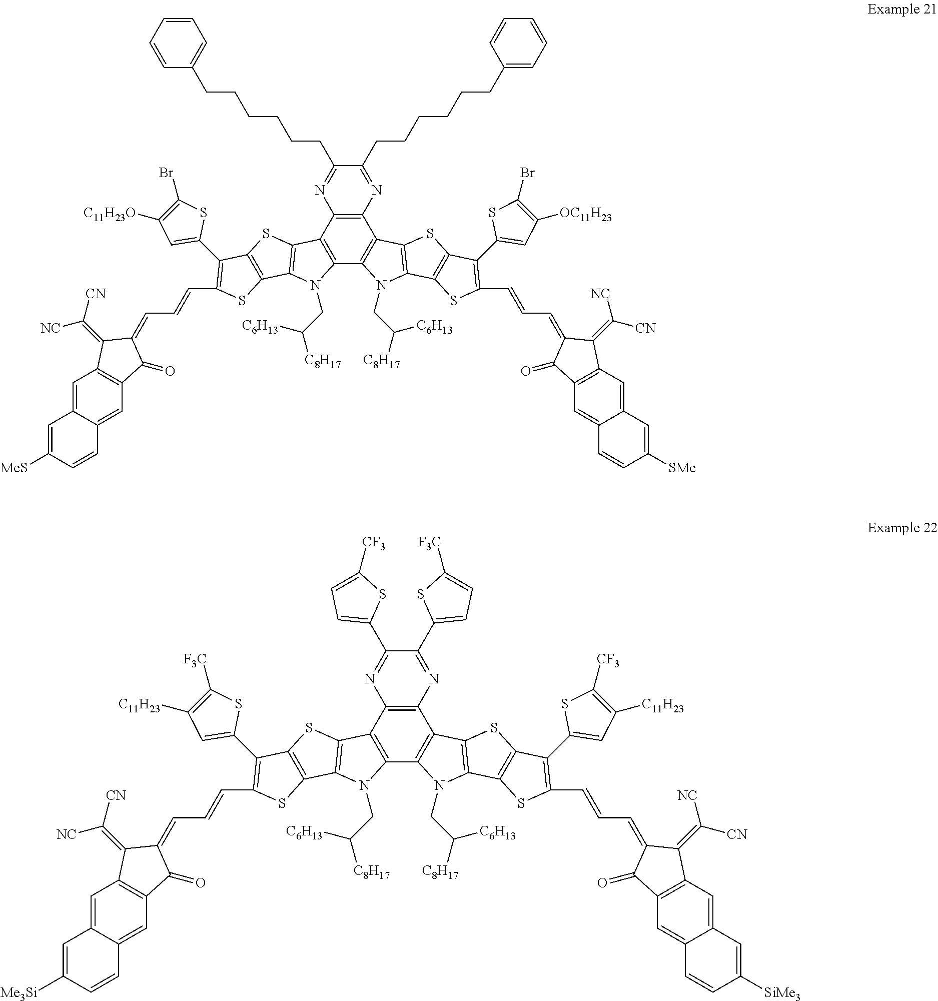

It should be understood that the above-listed embodiments are only intended to allow the person skilled in the art to understand the structure and composition of the present invention more clearly, and are not limited thereto.

In an embodiment, an organic composition of the present invention comprising at least one P-type organic semiconductor material and at least one N-type organic semiconductor material. The P-type organic semiconductor material comprises at least one of organic conjugated polymer or organic conjugated small molecule. The N-type organic semiconductor material comprises at least one organic material aforementioned.

Wherein, the P-type organic semiconductor material is further selected from at least one organic conjugated polymer. The conjugated polymer is composed of a plurality of monomers, and the monomers include one selected from the following structures and combinations thereof:

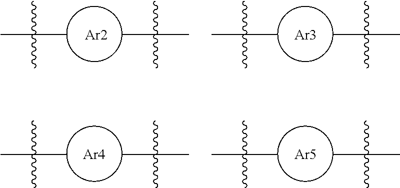

wherein Ar2, Ar3, Ar4 and Ar5 are independently selected from a monocyclic or polycyclic structure.

The conjugated polymer further comprises the following structures:

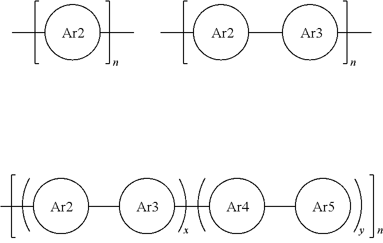

Wherein, Ar2, Ar3, Ar4 and Ar5 are monocyclic or polycyclic structures containing C4-C30 ring atoms respectively. n is a positive integer from 1 to 1000. x and y are molar fractions, where 0<x<1, 0<y<1 and x+y=1. In a preferred embodiment, at least one of the ring atoms included in Ar2, Ar3, Ar4 and Ar5 is a heteroatom, wherein the heteroatom is independently selected from at least one of S, O, Se, N, F, Cl and Si.

In one embodiment, Ar2 and Ar4 are independently selected from the following structures:

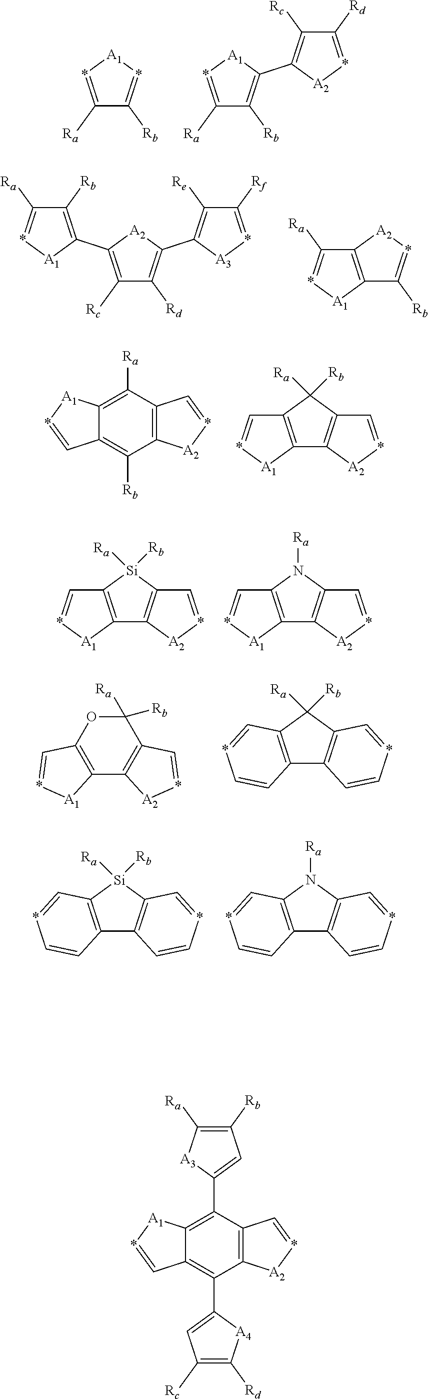

Wherein, each structure is connected by *. A₁, A₂, A₃ and A₄ are independently selected from O, S and Se. Rₐ, R_{b}, R_{c}, R_{d}, Rₑ and R_{f} are independently selected from the following groups: hydrogen, halogen, cyano group, C1-C30 alkyl with or without substituents, C1-C30 alkoxy with or without substituents, C1-C30 alkylthio with or without substituents, C1-C30 haloalkyl with or without substituents, C2-C30 ester with or without substituents, C1-C30 alkylaryl with or without substituents, C1-C30 alkyl heteroaryl with or without substituents, C1-C30 silyl aryl with or without substituents, C1-C30 silyl heteroaryl with or without substituents, C1-C30 alkoxyaryl with or without substituents, C1-C30 alkyloxy heteroaryl with or without substituents, C1-C30 alkylthioaryl with or without substituents, C1-C30 alkylthio heteroaryl with or without substituents, C1-C30 haloalkyl aryl with or without substituents, C1-C30 haloalkyl heteroaryl with or without substituents, C2-C30 esteryl aryl with or without substituents and C2-C30 esteryl heteroaryl with or without substituents.

In a preferred embodiment, Ar2 and Ar4 are independently selected from one of the following structures:

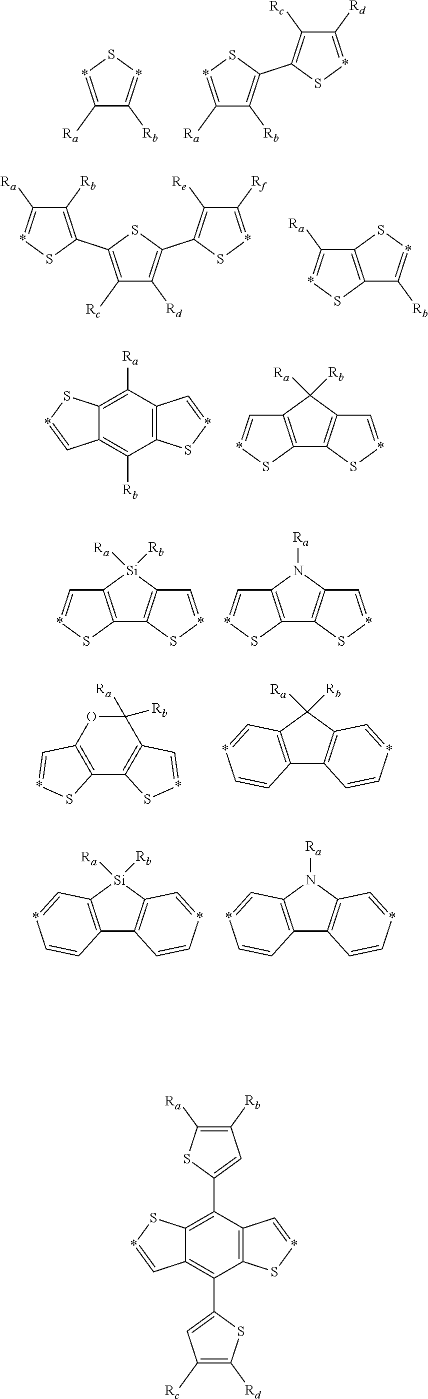

From the above embodiments, Ar2 and Ar4 are independently and preferably selected from one of the following structures:

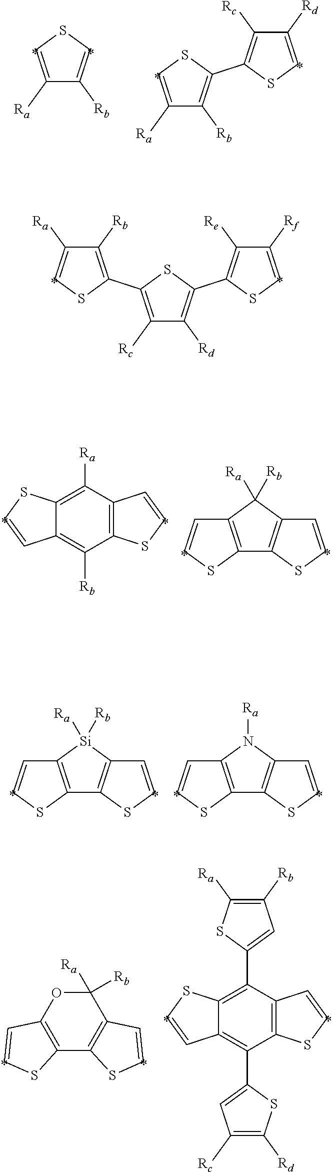

In one embodiment, Ar3 and Ar5 are independently selected from the following structures:

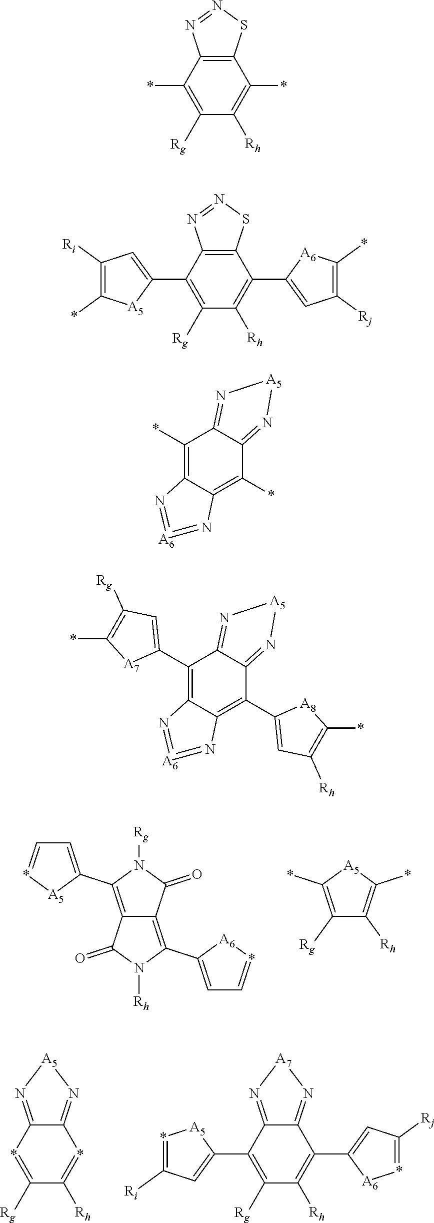

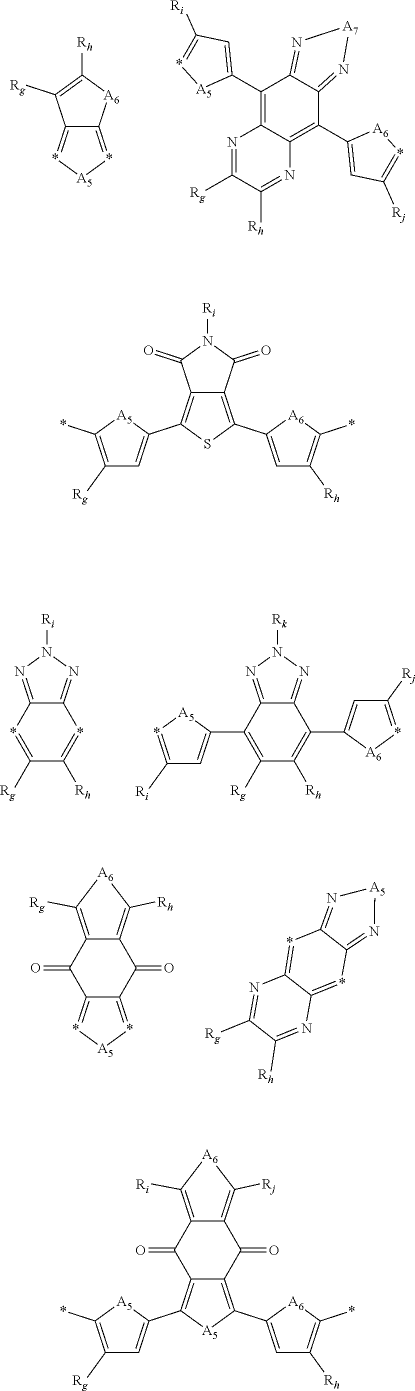

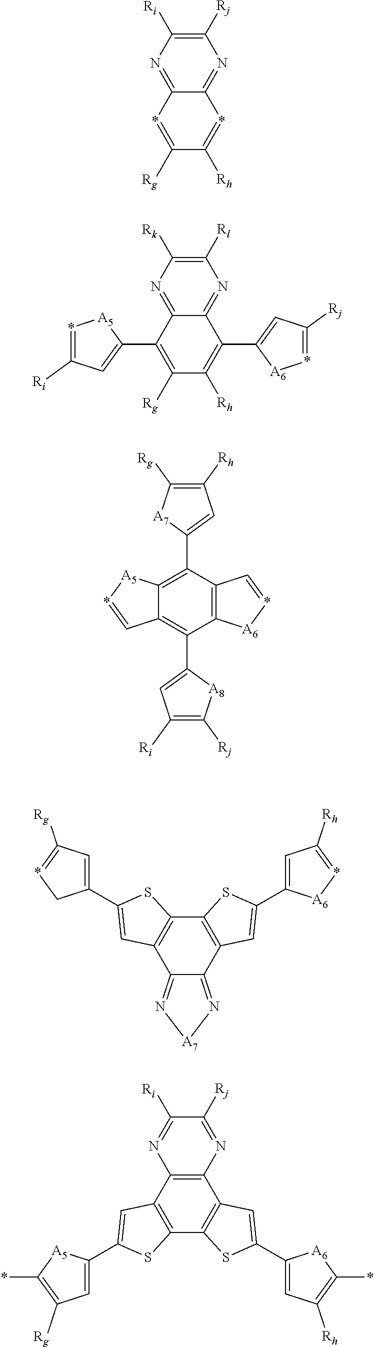

Wherein, each structure is connected by *. A₅, A₆, A₇ and A₈ are independently selected from O, S and Se. R_{g}, Rₕ, Rᵢ, Rⱼ, Rₖ and Rₗ are independently selected from the following groups: hydrogen, halogen, cyano group, C1-C30 alkyl with or without substituents, C1-C30 alkoxy with or without substituents, C1-C30 alkylthio with or without substituents, C1-C30 haloalkyl with or without substituents, C2-C30 ester with or without substituents, C1-C30 alkylaryl with or without substituents, C1-C30 alkyl heteroaryl with or without substituents, C1-C30 silyl aryl with or without substituents, C1-C30 silyl heteroaryl with or without substituents, C1-C30 alkoxyaryl with or without substituents, C1-C30 alkyloxy heteroaryl with or without substituents, C1-C30 alkylthioaryl with or without substituents, C1-C30 alkylthio heteroaryl with or without substituents, C1-C30 haloalkyl aryl with or without substituents, C1-C30 haloalkyl heteroaryl with or without substituents, C2-C30 esteryl aryl with or without substituents and C2-C30 esteryl heteroaryl with or without substituents. Wherein, * and * are bonded by a single band.

In a preferred embodiment, Ar3 and Ar5 are independently selected from one of the following structures:

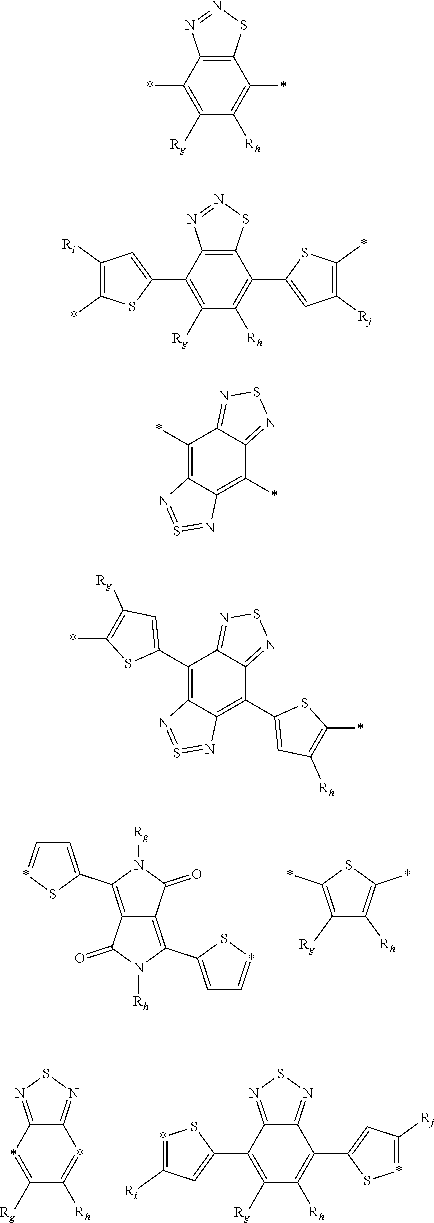

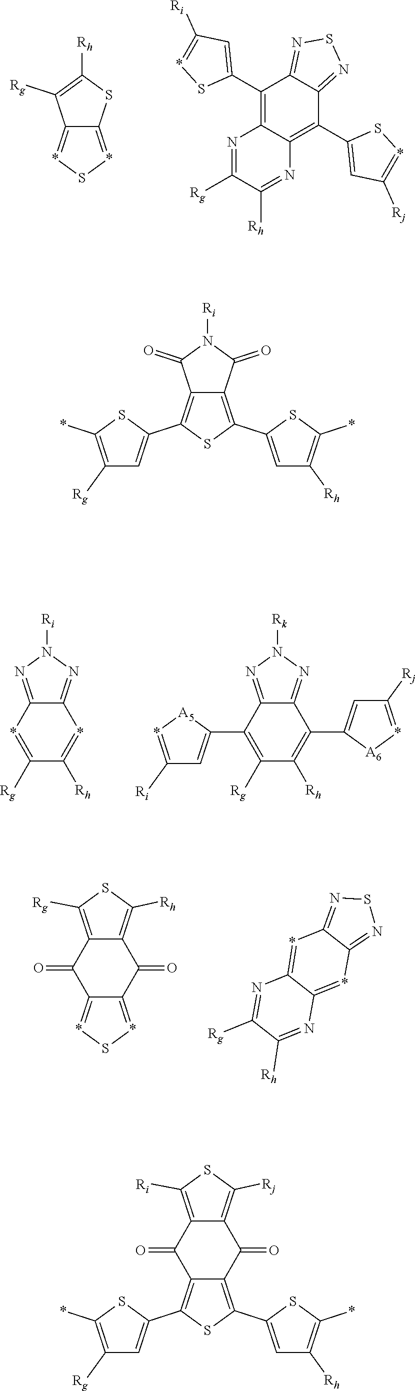

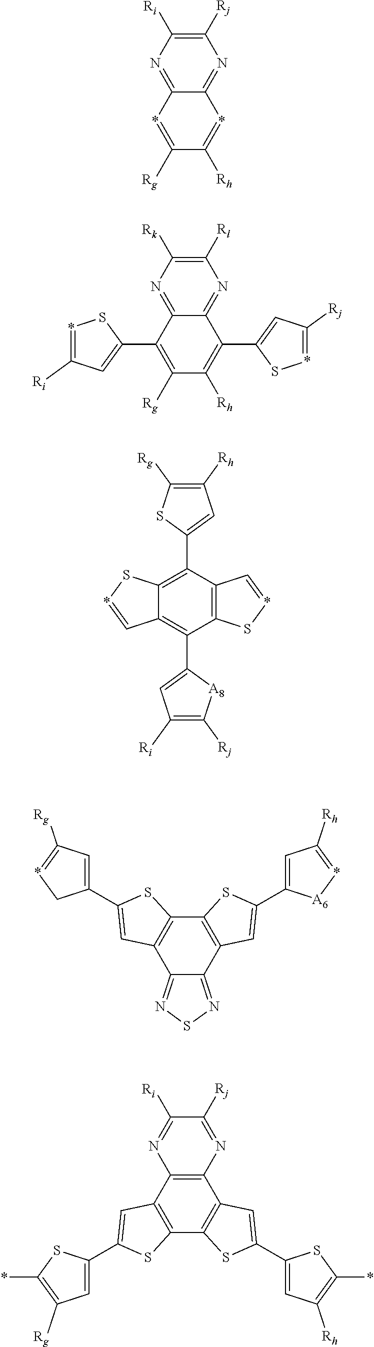

From the above embodiments, Ar3 and Ar5 are independently and preferably selected from one of the following structures:

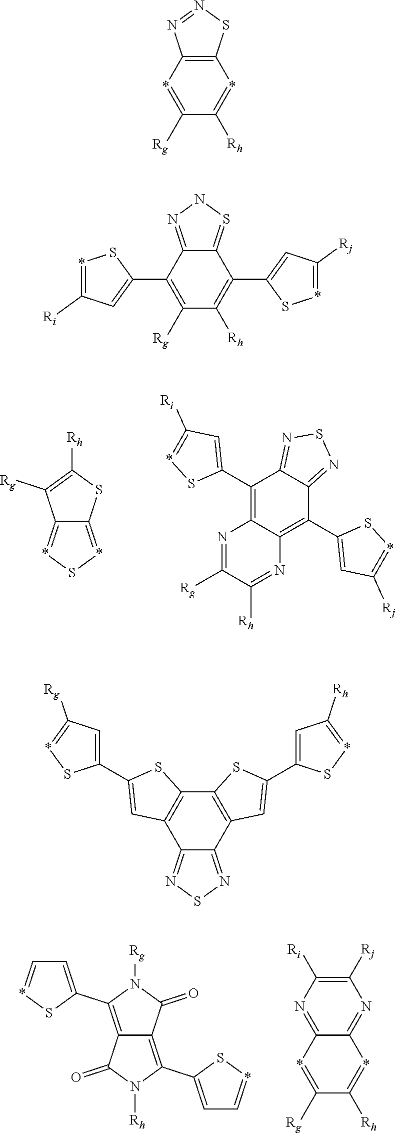

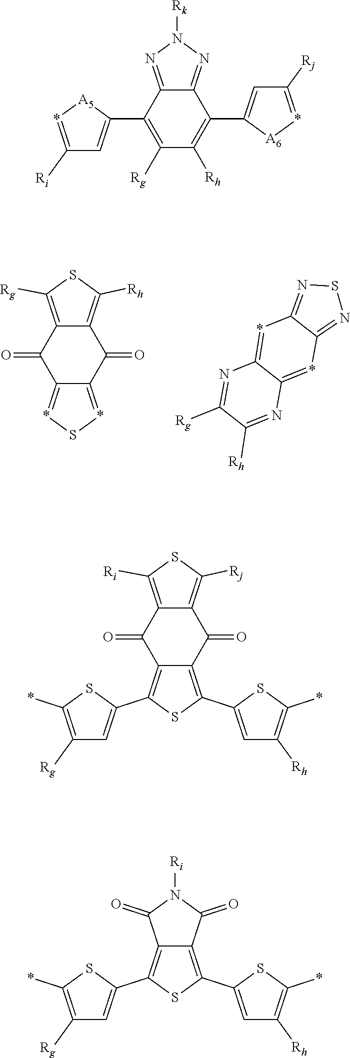

The substituents mentioned above can be independently selected from the following groups and their derivatives: C1-C30 alkyl, C3-C30 branched alkyl, C1-C30 silyl, C2-C30 ester, C1-C30 alkoxy, C1-C30 alkylthio, C1-C30 haloalkyl, C2-C30 olefin, C2-C30 alkyne, C2-C30 carbon chains containing cyano group, C1-C30 carbon chains containing nitro groups, C1-C30 carbon chains containing hydroxy groups, C3-C30 carbon chains containing keto groups, halogens, cyano groups, hydrogen, deuterium and tritium. The above-mentioned aryl group and heteroaryl group may have a monocyclic or polycyclic structure.

In practice, the conjugated polymer further comprises the following embodiments P-1˜P-39 and PBDB-T:

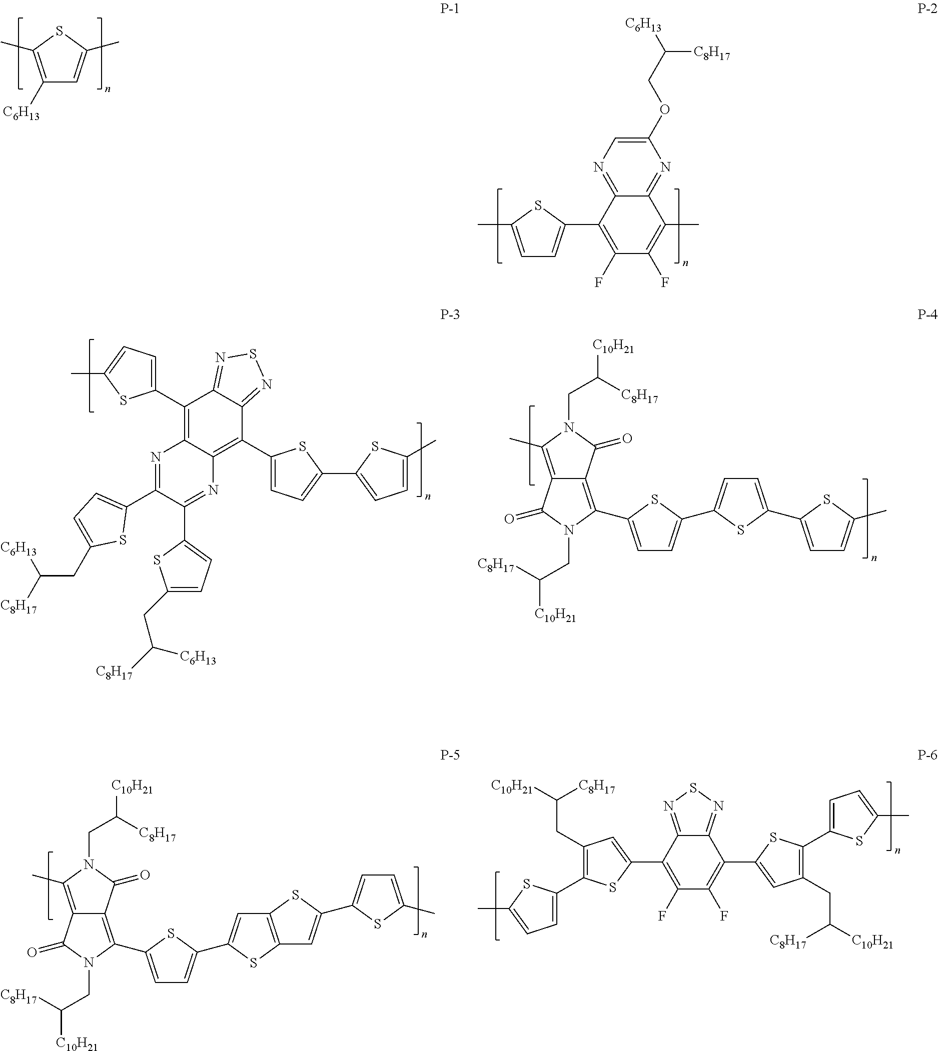

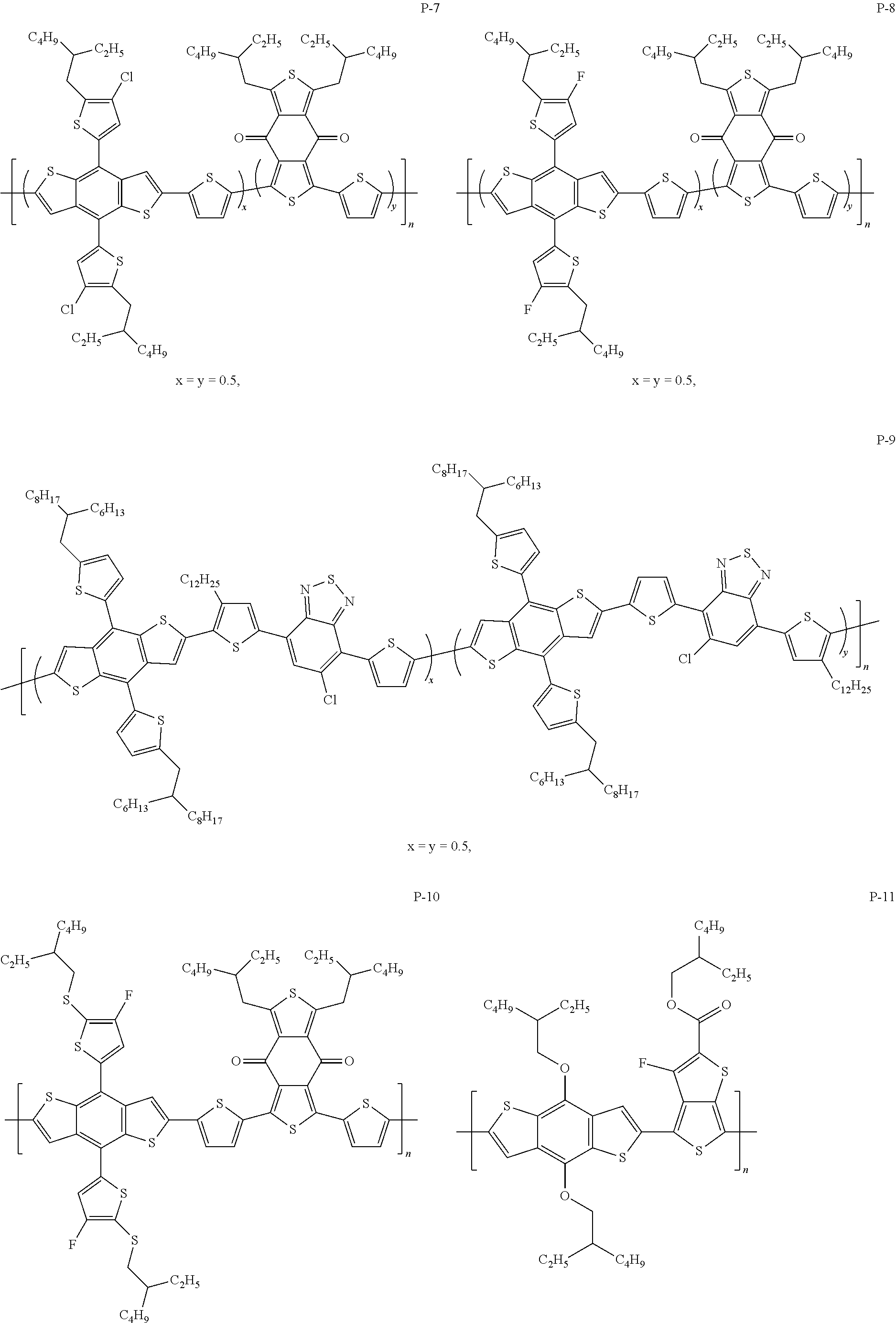

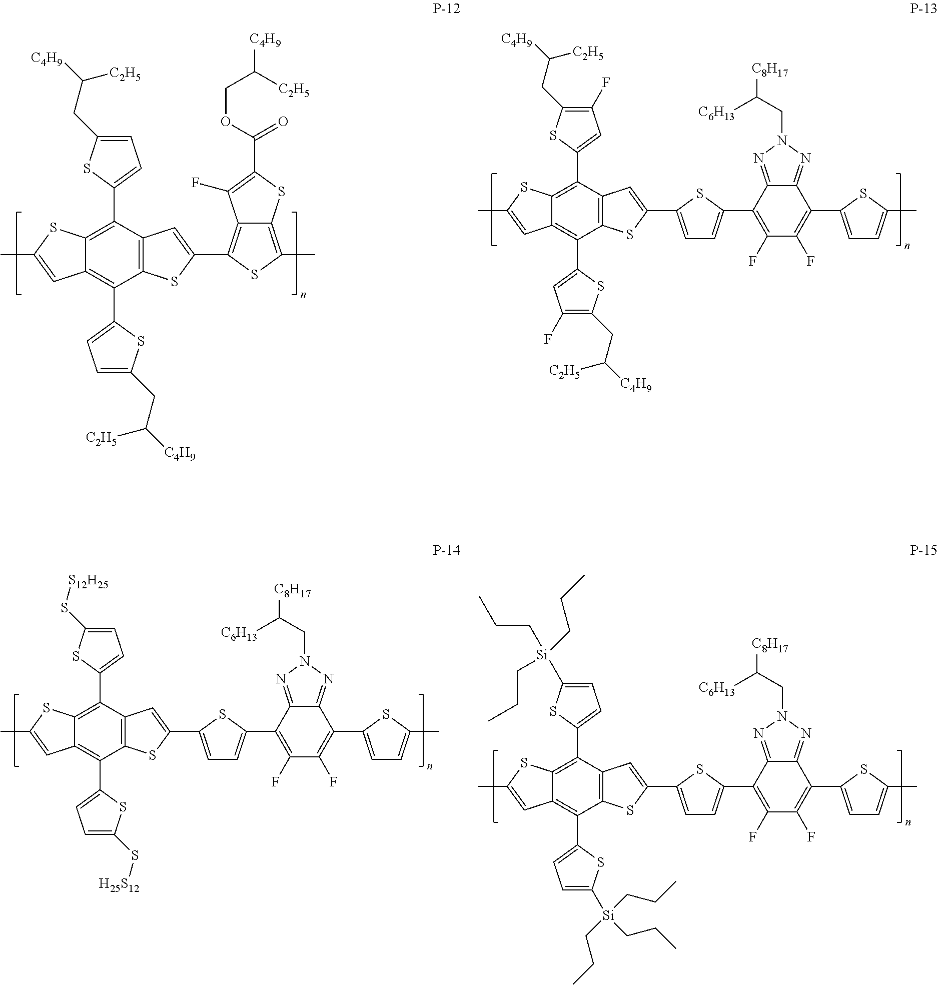

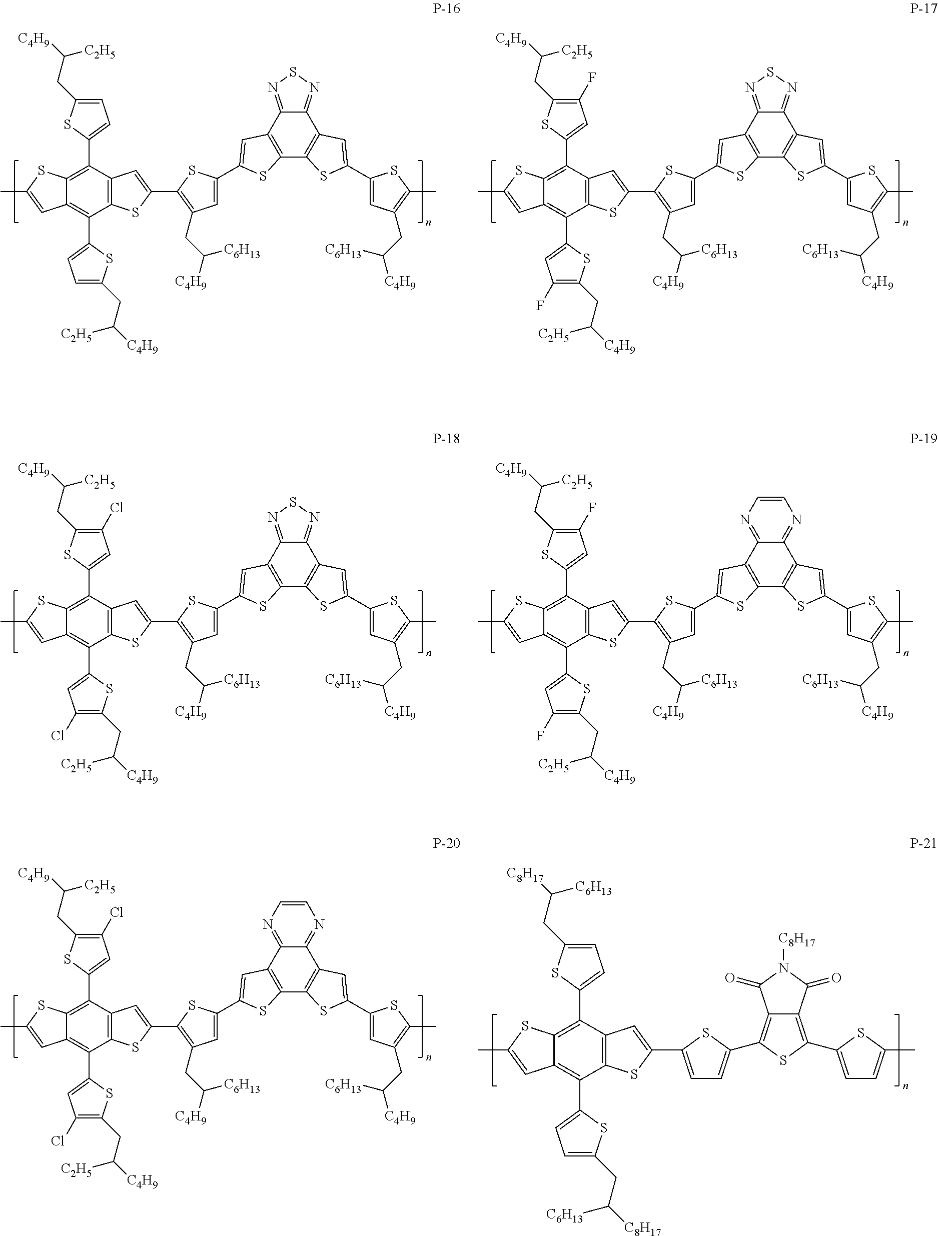

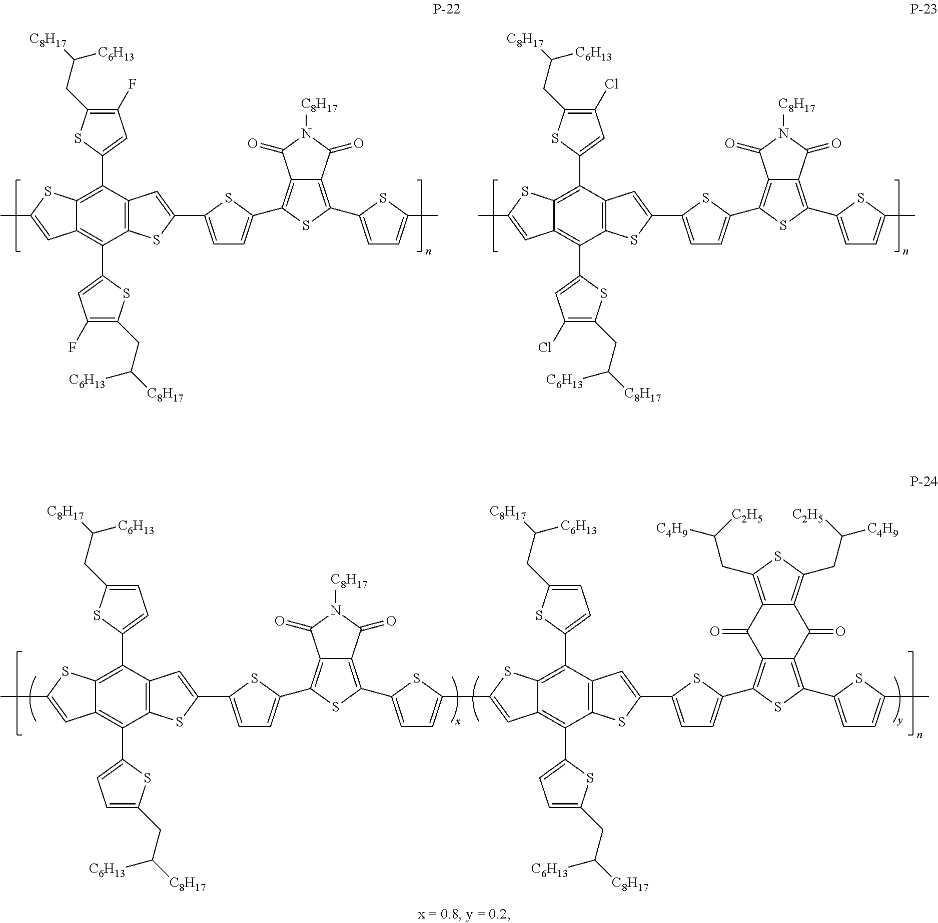

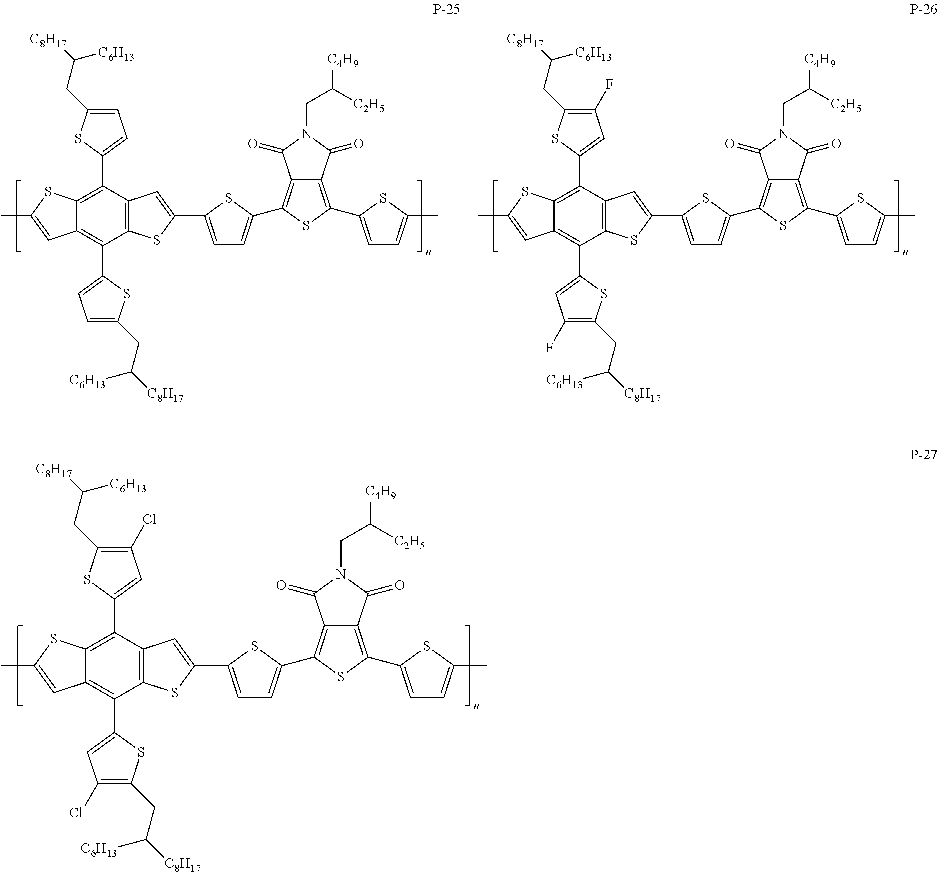

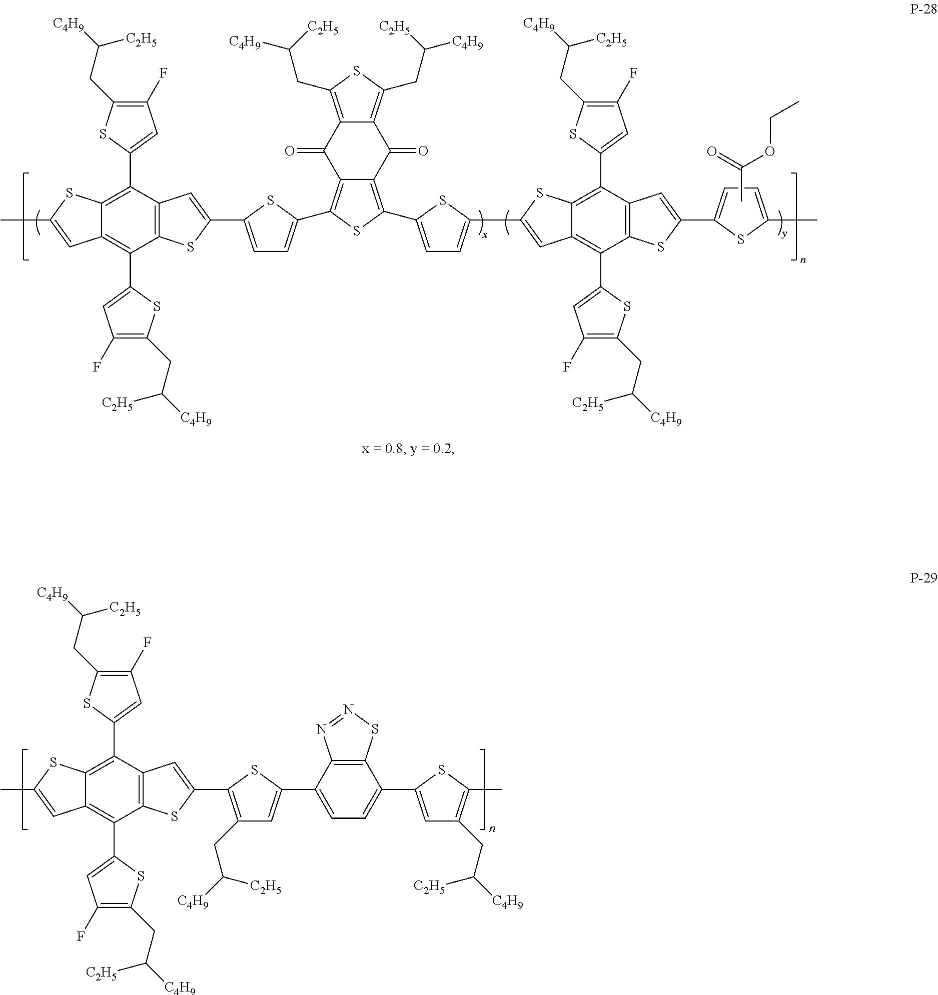

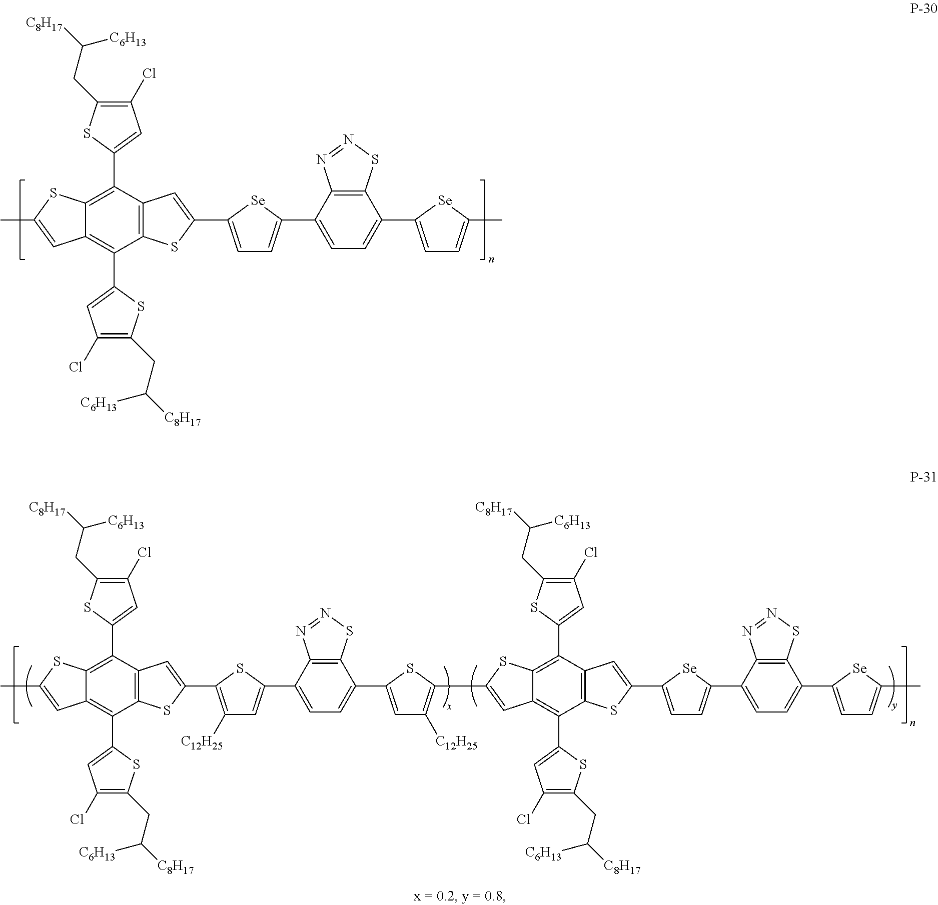

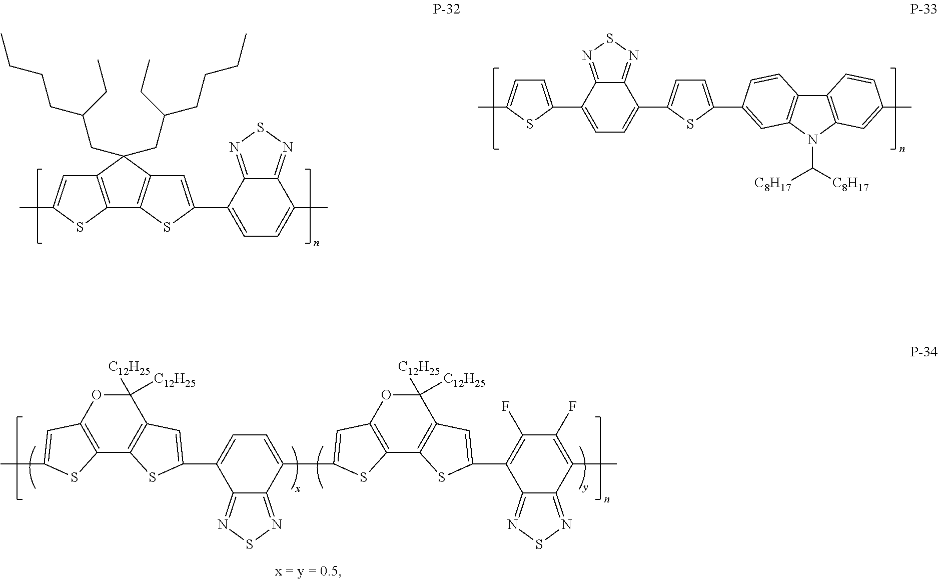

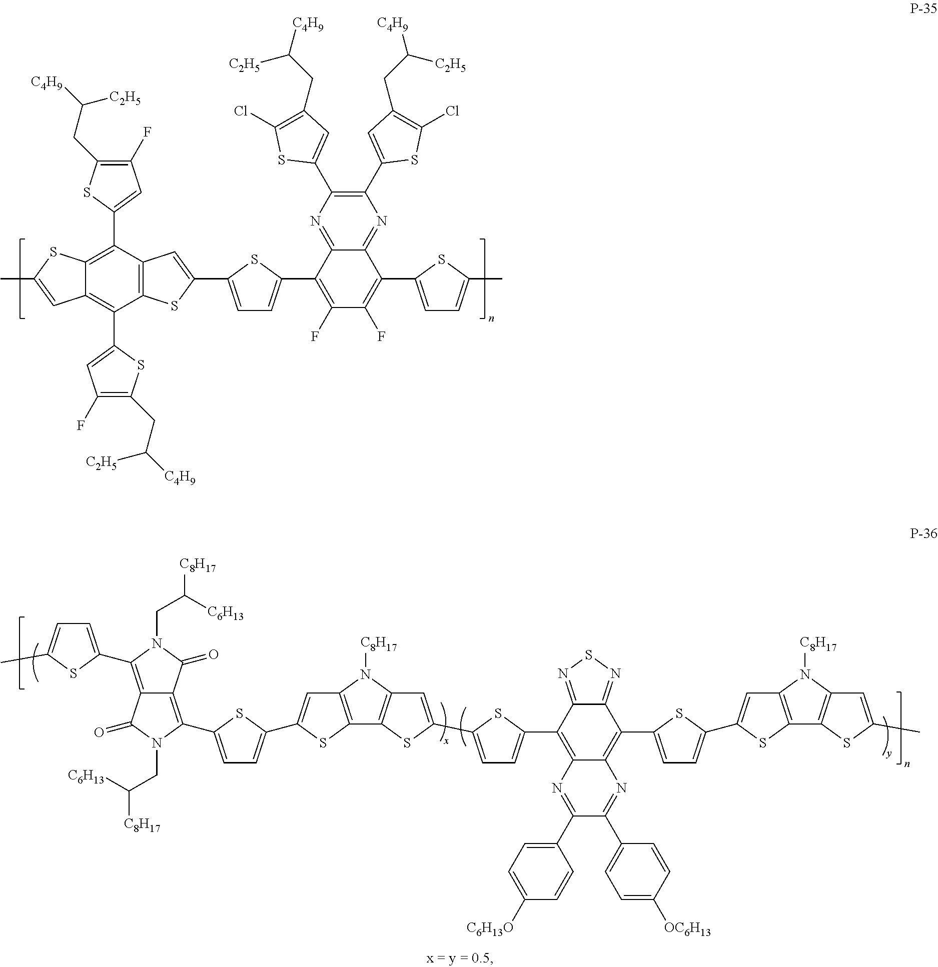

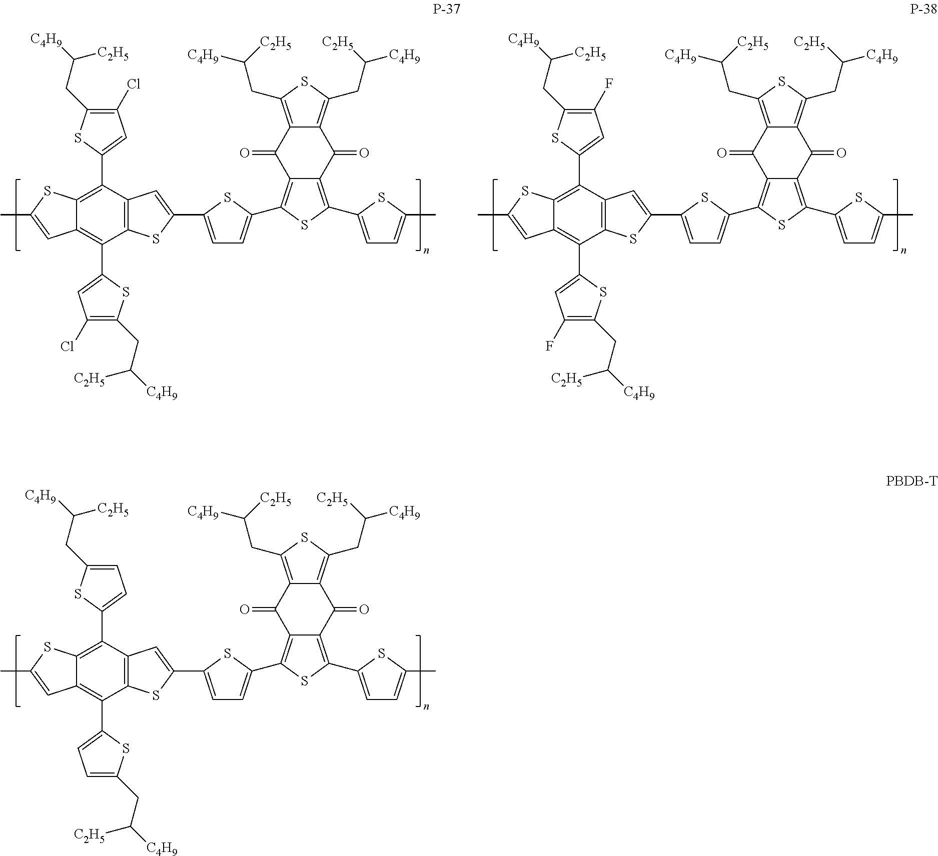

It should be understood that the above-listed embodiments are only intended to allow the person skilled in the art to understand the structure and composition of the present invention more clearly, and are not limited thereto.

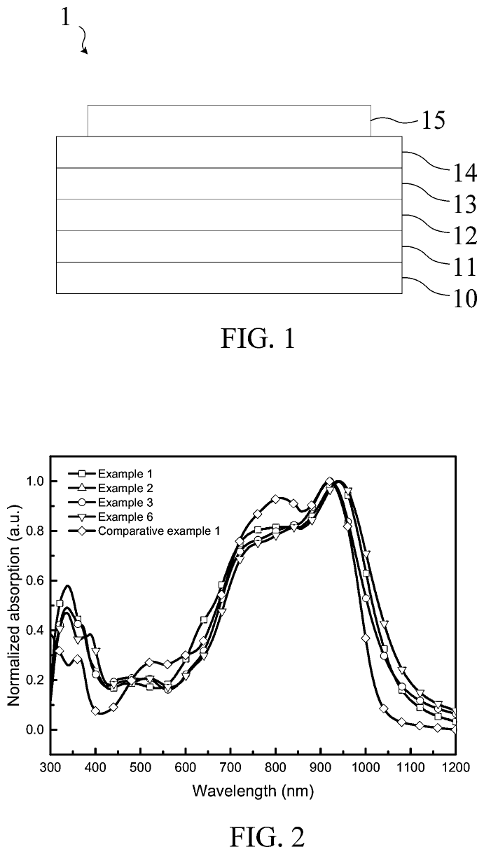

Please refer to FIG. 1. FIG. 1 shows a schematic structural diagram of one embodiment of an organic optoelectronic device of the present invention. As shown in FIG. 1, in another embodiment, the present invention further provides an organic optoelectronic device 1, which comprises a first electrode 11, a second electrode 15 and an active layer 13. The active layer 13, which comprises at least one of the aforementioned organic material comprising Formula I and the aforementioned organic composition, is disposed between the first electrode 11 and the second electrode 15. The organic optoelectronic device 1 further comprises a first carrier transporting layer 12 and a second carrier transporting layer 14. The organic optoelectronic device 1 may have a stacked structure, which sequentially includes a substrate 10, the first electrode 11 (transparent or semi-transparent electrode), the first carrier transporting layer 12, the active layer 13, the second carrier transporting layer 14 and the second electrode 15. The first carrier transporting layer 12 is configured to transport carriers in the first electrode 11 and the active layer 13, and the second carrier transporting layer 14 is configured to transport carriers in the active layer 13 and the second electrode 15. Specifically, the first carrier transporting layer 12 is one of an electron transporting layer and a hole transporting layer, and the second carrier transporting layer 14 is the other one. In detail, when the first carrier transporting layer 12 is the electron transporting layer, the second carrier transporting layer 14 is the hole transporting layer, which is an inverted stacked structure; when the first carrier transporting layer 12 is the hole transporting layer, the second carrier transporting layer 14 is an electron transporting layer, which is a conventional stacked structure. In practice, the organic optoelectronic device 1 may comprise an organic photovoltaic device, an organic photodetector device, or an organic light emitting diode.

In order to illustrate the organic composition of the present invention more clearly, the following experiments will be conducted using Comparative Example 1 and organic material Examples 1-3 and Example 6 of the invention to illustrate the differences in efficacy. These materials will then be further utilized as N-type organic semiconductor materials combined with at least one P-type organic semiconductor material to prepare the organic composition. The active layers comprising the aforementioned organic materials or organic compositions will be fabricated into organic optoelectronic devices for material testing and device performance evaluation.

For the optical physical quality testing part of material testing and device testing, the UV absorption spectrum measurement instrument model is Hitachi UH5700, and the oxidation potential is measured by using cyclic voltammetry with CH Instrument 611E.

### Synthesis of Example 1

### Synthesis of M2

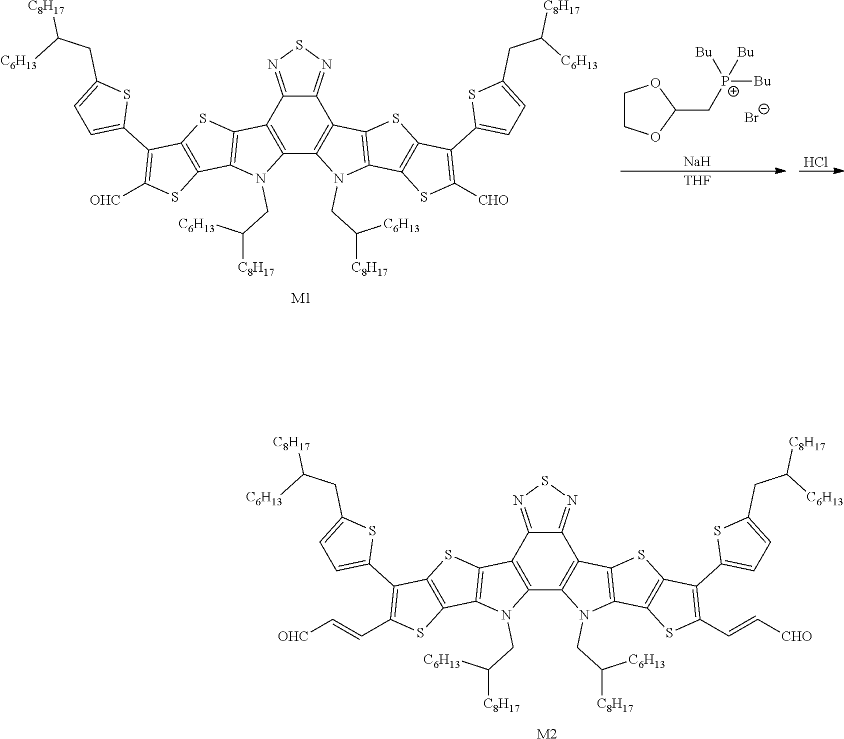

Tributyl(1,3-dioxolan-2-ylmethyl)phosphonium bromide (0.66 g, 1.80 mmol), M1 (0.70 g, 0.45 mmol) and sodium hydride (60%, 0.10 g, 2.70 mmol) were placed sequentially into a 100 mL two necked flask. Under argon atmosphere, anhydrous tetrahydrofuran was added and stirred with a magnet, and the mixture was reacted at room temperature for 6 hours. 10% dilute hydrochloric acid (3.5 mL) was added, and the mixture was stirred at room temperature for 30 minutes. The mixture was extracted three times with heptane/water, the organic layers were collected, magnesium sulfate was added to remove water, and the solvent was removed. The crude product was purified by silica gel column chromatography (the eluent was heptane/dichloromethane=1/3) to obtain a red oil M2 (700 mg, yield 97%). ¹H NMR (600 MHz, CDCl₃): δ 9.70 (d, J=7.8 Hz, 2H), 8.09 (d, J=15.0 Hz, 2H), 7.31 (d, J=4.2 Hz, 2H), 6.94 (d, J=3.6 Hz, 2H), 6.60 (dd, J=15.0 Hz, J=7.8 Hz, 2H) 4.62 (d, J=4.2, 4H), 2.89 (t, J=7.8, 4H), 2.06 (m, 2H), 2.78 (m, 2H), 1.37-0.67 (m, 120H).

### Synthesis of Example 1

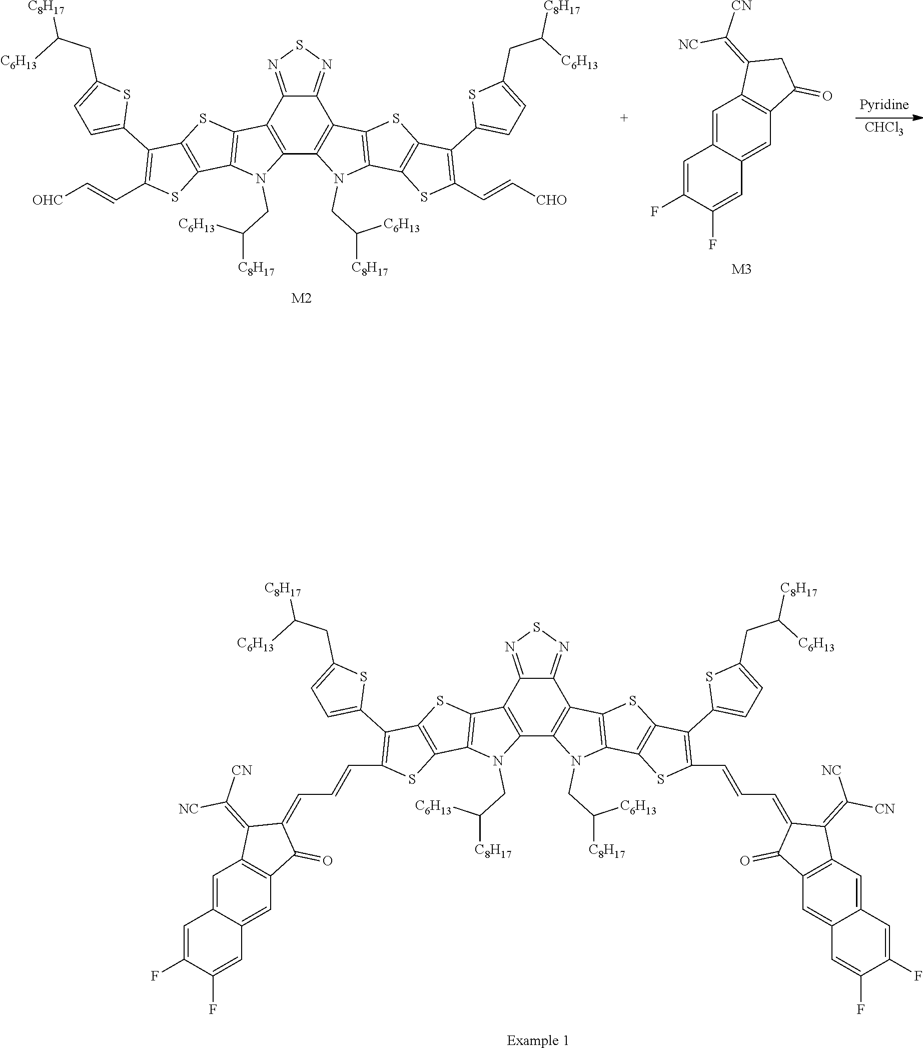

M2 (350 mg, 0.22 mmol), M3 (183 mg, 0.65 mmol) and chloroform (10.5 mL) were placed in a 100 mL two necked flask and stirred with a magnet under argon for 30 minutes. Pyridine (0.18 mL) was added in an ice bath and the reaction was allowed to react for 24 hours. Methanol was added to precipitate the product, and the solid was collected by suction filtration. The crude product was purified by silica gel column chromatography (the eluent was heptane/dichloromethane=1/5) to obtain Example 1 (320 mg, yield 69%) as a blue-black solid. ¹H NMR (600 MHz, CDCl₃): δ 9.11 (s, 2H), 8.89 (dd, J=14.4 Hz, J=12.0 Hz, 2H), 8.60 (d, J=11.4, 2H), 8.30 (s, 2H), 8.08 (d, J=14.4 Hz, 2H), 7.87-7.80 (m, 4H), 7.36 (d, J=3.6 Hz, 2H), 6.98 (d, J=3.6 Hz, 2H), 4.69 (d, J=7.2 Hz, 4H), 2.90 (d, J=6.6 Hz, 4H), 2.13-2.11 (m, 2H), 1.78 (m, 2H), 1.38-0.70 (m, 120H).

### Synthesis of Example 2

### Synthesis of M5

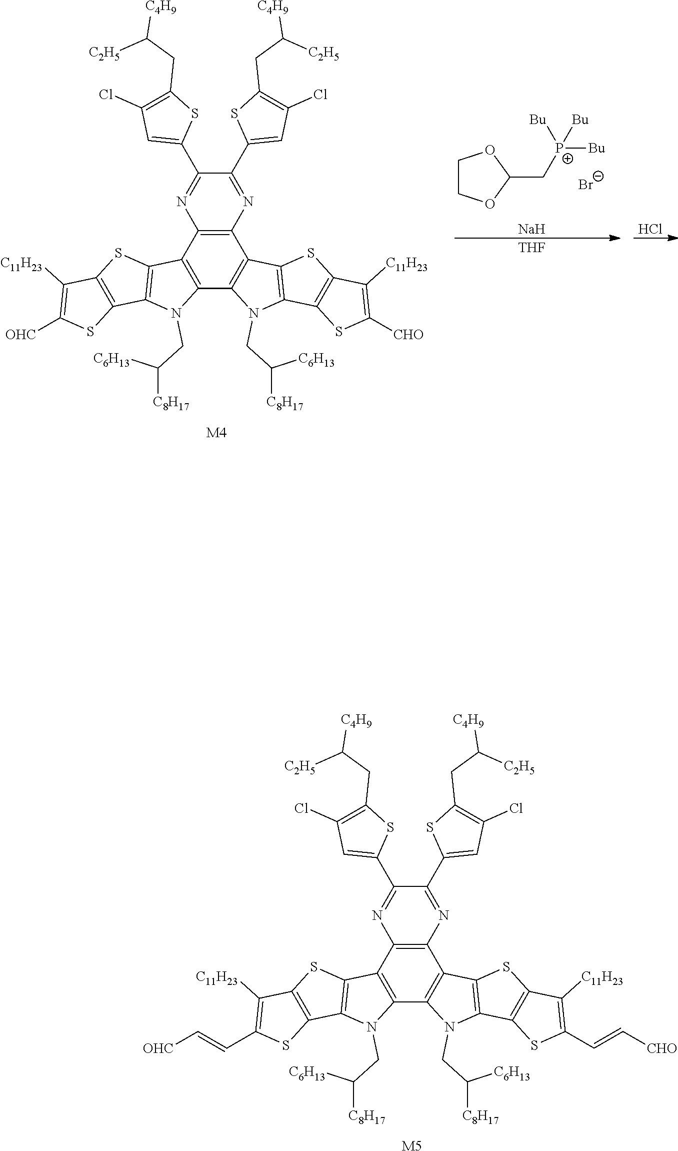

Tributyl(1,3-dioxolan-2-ylmethyl)phosphonium bromide (0.58 g, 1.56 mmol), M4 (0.66 g, 0.39 mmol) and sodium hydride (60%, 0.06 g, 2.34 mmol) were placed sequentially into a 100 mL two necked flask. Under argon atmosphere, anhydrous tetrahydrofuran was added and stirred with a magnet, and the mixture was reacted at room temperature for 6 hours. 10% dilute hydrochloric acid (3.3 mL) was added, and the mixture was stirred at room temperature for 30 minutes. The mixture was extracted three times with heptane/water, the organic layers were collected, magnesium sulfate was added to remove water, and the solvent was removed. The crude product was purified by silica gel column chromatography (the eluent was heptane/dichloromethane=1/1) to obtain a red oil M5 (475 mg, yield 69%). ¹H NMR (600 MHz, CDCl₃): δ 9.70 (d, J=7.8 Hz, 2H), 7.78 (d, J=15.0 Hz, 2H), 7.33 (s, 2H), 6.53 (dd, J=15.3 Hz, J=7.5 Hz, 2H), 4.63 (d, J=6.6 Hz, 4H), 3.02 (t, J=7.8, 4H), 2.88 (m, 4H), 2.07 (m, 2H), 1.78 (m, 2H), 1.71 (m, 2H), 1.47-0.69 (m, 128H).

### Synthesis of Example 2

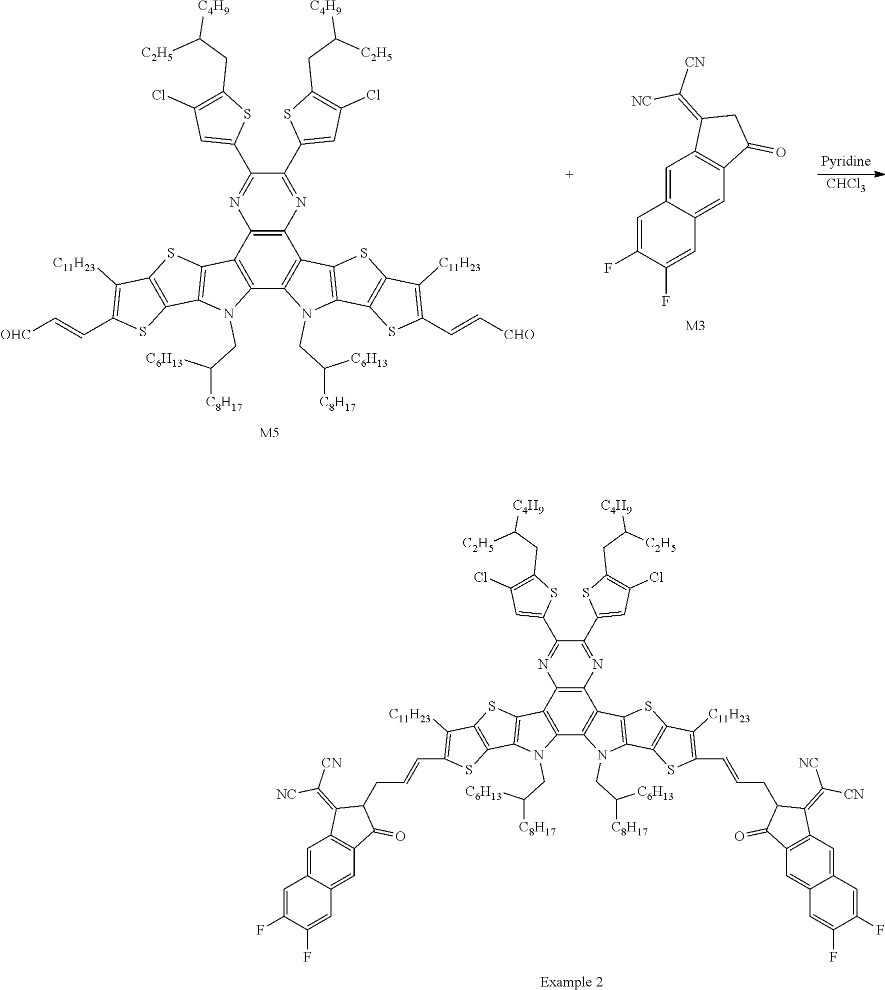

M4 (100 mg, 0.06 mmol), M3 (48 mg, 0.17 mmol) and chloroform (5 mL) were placed in a 100 mL two necked flask and stirred with a magnet under argon for 30 minutes. Pyridine (0.1 mL) was added in an ice bath and the reaction was allowed to react for 24 hours. Methanol was added to precipitate the product, and the solid was collected by suction filtration. The crude product was purified by silica gel column chromatography (the eluent was heptane/dichloromethane=1/10) to obtain Example 2 (60 mg, yield 45%) as a blue-black solid. ¹H NMR (600 MHZ, CDCl₃): δ 9.09 (s, 2H), 8.81-8.77 (m, 2H), 8.60 (d, J=11.4 Hz, 2H), 8.27 (s, 2H), 7.84-7.77 (m, 6H), 7.35 (s, 2H), 4.69 (m, 4H), 3.08 (t, J=7.8, 4H), 2.89 (m, 4H), 2.18 (m, 2H), 1.89 (m, 4H), 1.82 (m, 2H), 1.43-0.72 (m, 126H).

### Synthesis of Example 3

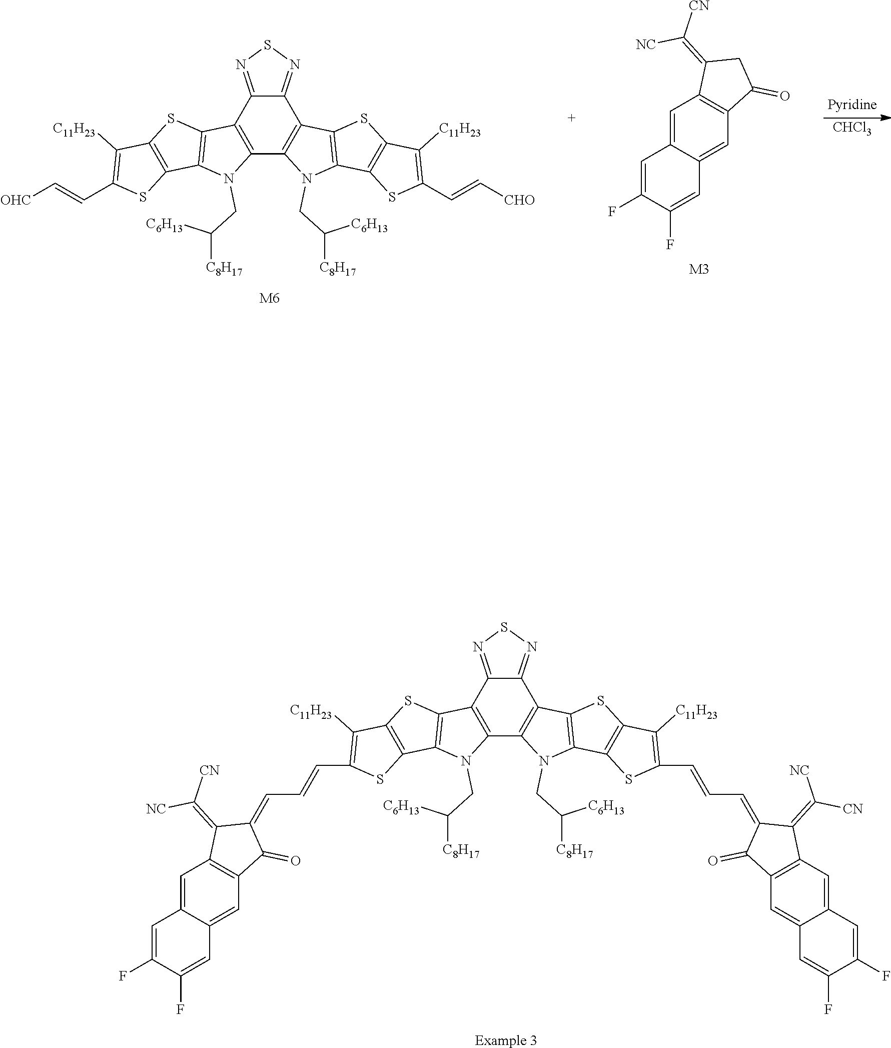

M6 (200 mg, 0.15 mmol), M3 (130 mg, 0.45 mmol) and chloroform (5 mL) were placed in a 100 mL two necked flask and stirred with a magnet under argon for 30 minutes. Pyridine (0.1 mL) was added in an ice bath and the reaction was allowed to react for 20 hours. Methanol was added to precipitate the product, and the solid was collected by suction filtration. The crude product was purified by silica gel column chromatography (the eluent was heptane/dichloromethane=1/10) to obtain Example 3 (270 mg, yield 99%) as a blue-black solid. ¹H NMR (500 MHz, CDCl₃): δ 9.12 (s, 2H), 8.83-8.78 (m, 2H), 8.62 (d, J=12.0 Hz, 2H), 8.30 (s, 2H), 7.87-7.81 (m, 4H), 7.78 (d, J=14.0 Hz, 2H), 4.66 (d, J=8.0 Hz, 4H), 3.05 (t, J=7.8 Hz, 4H), 2.12-2.11 (m, 2H), 1.90-1.85 (m, 4H), 1.30-0.71 (m, 98H)

### Synthesis of Example 6

### Synthesis of M14

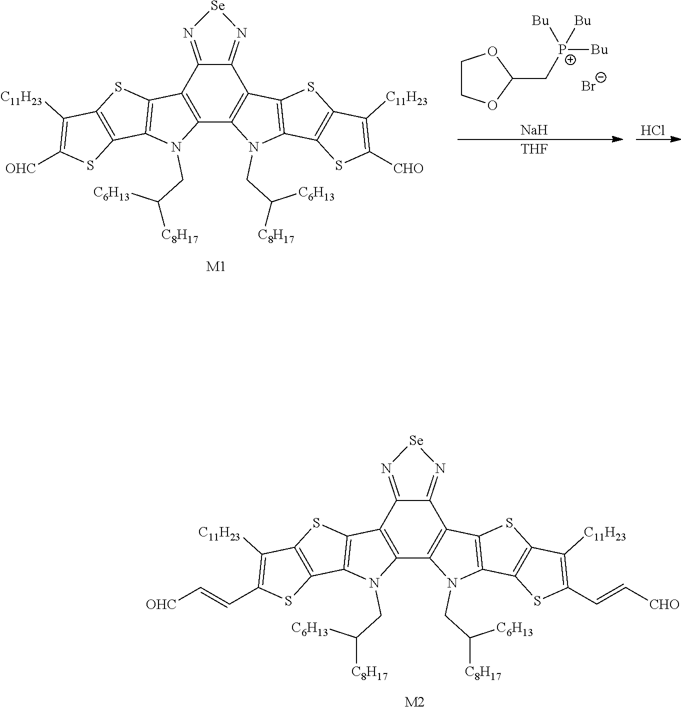

Tributyl(1,3-dioxolan-2-ylmethyl)phosphonium bromide (0.34 g, 0.92 mmol), M13 (0.30 g, 0.23 mmol) and sodium hydride (60%, 0.03 g, 1.39 mmol) were placed sequentially into a 100 mL two necked flask. Under argon atmosphere, anhydrous tetrahydrofuran was added and stirred with a magnet, and the mixture was reacted at room temperature for 6 hours. 10% dilute hydrochloric acid (1.5 mL) was added, and the mixture was stirred at room temperature for 30 minutes. The mixture was extracted three times with heptane/water, the organic layers were collected, magnesium sulfate was added to remove water, and the solvent was removed. The crude product was purified by silica gel column chromatography (the eluent was heptane/dichloromethane=1/1.5) to obtain a red oil M14 (260 mg, yield 83%). ¹H NMR (600 MHz, CDCl₃): δ 9.70 (d, J=7.5 Hz, 2H), 7.77 (d, J=15.0 Hz, 2H), 6.51 (dd, J=7.5 Hz, J=15.0 Hz, 2H), 4.55 (d, J=8.0 Hz, 4H), 2.98 (t, J=7.5 Hz, 4H), 2.04 (m, 2H), 1.86-1.83 (m, 4H), 1.28-0.67 (m, 98H).

### Synthesis of Example 6

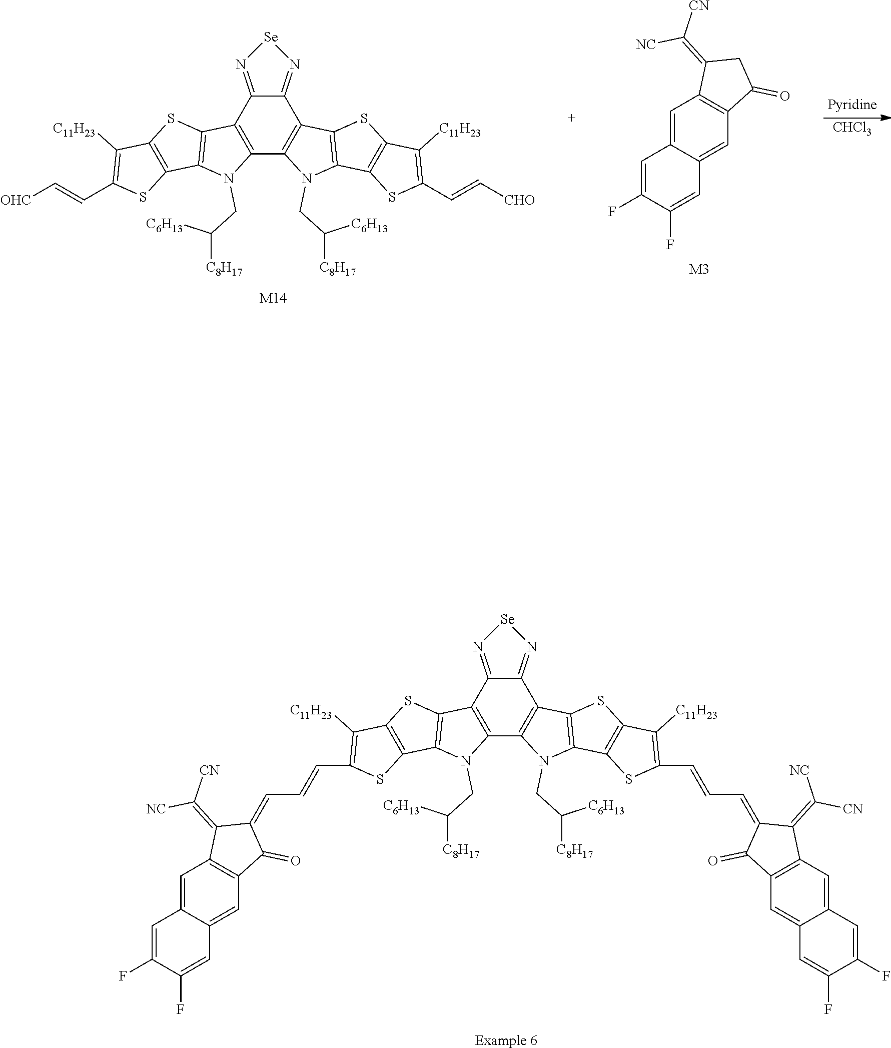

M14 (260 mg, 0.19 mmol), M3 (161 mg, 0.58 mmol) and chloroform (8 mL) were placed in a 100 mL two necked flask and stirred with a magnet under argon for 30 minutes. Pyridine (0.26 mL) was added in an ice bath and the reaction was allowed to react for 24 hours. Methanol was added to precipitate the product, and the solid was collected by suction filtration. The crude product was purified by silica gel column chromatography (the eluent was heptane/dichloromethane=1/9) to obtain Example 6 (227 mg, yield 63%) as a blue-black solid. ¹H NMR (600 MHz, CDCl₃): δ 9.12 (s, 2H), 8.82-8.78 (m, 2H), 8.62 (d, J=12.0 Hz, 2H), 8.29 (s, 2H), 7.87-7.77 (m, 6H), 4.61 (d, J=7.2 Hz, 4H), 3.04 (t, J=7.8 Hz, 4H), 2.12 (m, 2H), 1.87 (quint, J=7.2 Hz, 4H), 1.52-0.71 (m, 98H).

Material testing of organic material Examples 1 to 3 and Example 6 and Comparative Example 1 includes material optical property testing:

The structure of Comparative Example 1 is as follows:

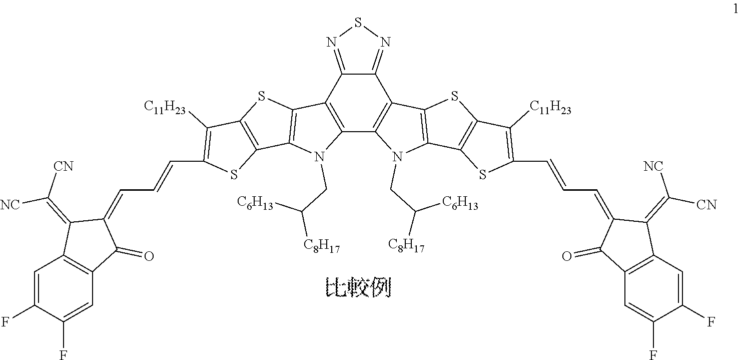

Please refer to FIG. 2 and Table 1. FIG. 2 shows absorption spectra in thin-film state of Comparative Example 1, Example 1, Example 2, Example 3, and Example 6 of the organic materials. Table 1 shows the material test of organic material Comparative Example 1, Example 1, Example 2, Example 3, and Example 6 (including the data results of FIG. 2).

TABLE 1
the material test of organic material Comparative Example 1, Example 1, Example
2, Example 3, and Example 6 (including the data results of FIG. 2).

| | | | | ε | | | |
|---|---|---|---|---|---|---|---|
| organic | λₛₒₗₙ^{max} | λ_{film}^{max} | λ_{film}^{onset} | (10⁵ | E_{g}^{opt} | HOMO | LUMO |
| material | (nm) | (nm) | (nm) | cm⁻¹M⁻¹) | (eV) | (eV) | (eV) |
| Comparative | 801 | 918 | 1025 | 1.19 | 1.21 | −5.51 | −4.30 |
| Example 1 | | | | | | | |
| Example 1 | 841 | 939 | 1069 | 1.35 | 1.16 | −5.44 | −4.28 |
| Example 2 | 847 | 900 | 1014 | 1.16 | 1.22 | −5.52 | −4.37 |
| Example 3 | 835 | 922 | 1058 | 1.38 | 1.17 | −5.54 | −4.37 |
| Example 6 | 844 | 943 | 1080 | 1.54 | 1.15 | −5.43 | −4.28 |

As shown in FIG. 2 and Table 1, the organic material Example 1, Example 2, Example 3 and Example 6 have good performance in absorption spectra. The maximum absorption values of the organic materials in thin-film state, as shown in Table 1, fall within the range of 900-943 nm, and the onset absorption values range from 1014-1080 nm. In FIG. 2, the thin film absorption spectrum of the organic material shows good absorption properties in the range of 300-1100 nm, with an extinction coefficient of 1.16-1.54×10⁵ cm⁻¹M⁻¹. The application range of the above-mentioned Comparative Example 1, Example 1, Example 2, Example 3, and Example 6 can be from visible light to infrared light.

Thermal stability performance test of single material absorbance:

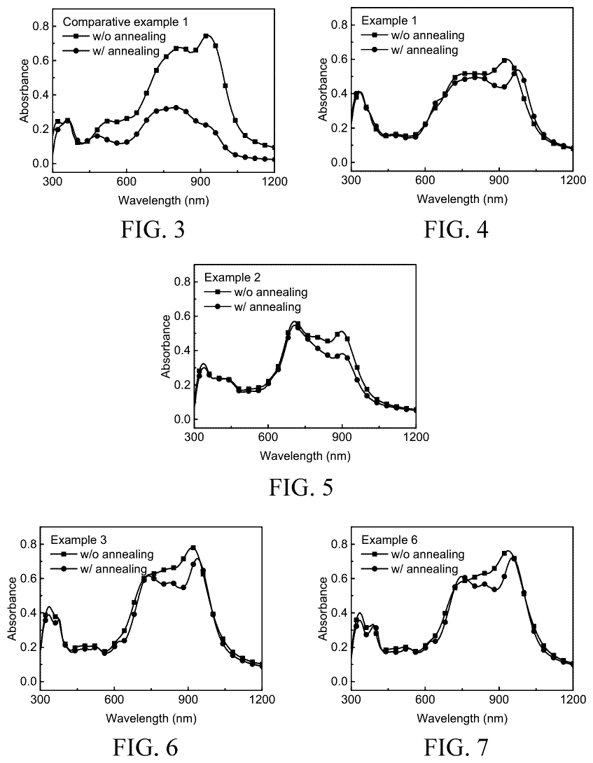

Please refer to FIG. 3 to FIG. 7. FIG. 3 shows the thin-film absorption spectra of Comparative Example 1 of the organic materials with and without annealing. FIG. 4 shows the thin-film absorption spectra of Example 1 of the organic materials with and without annealing. FIG. 5 shows the thin-film absorption spectra of Example 2 of the organic materials with and without annealing. FIG. 6 shows the thin-film absorption spectra of Example 3 of the organic materials with and without annealing. FIG. 7 shows the thin-film absorption spectra of Example 6 of the organic materials with and without annealing. In order to test the stability of the material after annealing, the thin film organic material was heated at 220° C. for 30 minutes in the atmosphere, and the absorption spectrum was used to observe the changes in its absorption intensity and waveform before and after annealing. As shown in FIG. 3, the absorption intensity of the organic material Comparative Example 1 decreases significantly after annealing, and the absorption spectrum undergoes a blue shift. As shown in FIG. 4 to FIG. 7, in contrast, the absorption intensity of organic material Example 1, Example 2, Example 3, and Example 6 does not decrease significantly after annealing, and maintains a certain onset absorption value. It can be seen that the organic materials of the present invention have good thermal stability and can maintain good device performance under device manufacturing and device operation requiring high temperature operation.

Preparation and performance testing of organic photodetectors of organic optoelectronic devices:

A glass coated by a pre-patterned indium tin oxides (ITO) with a sheet resistance of ˜15 Ω/sq is used as a substrate. The substrate is ultrasonically oscillated in soap deionized water, deionized water, acetone, and isopropanol in sequence, and washed in each step for 15 minutes. The washed substrate is further treated with a UV-ozone cleaner for 15 minutes. The top coat of AZO (Aluminum-doped zinc oxide) solution is spin coated on the ITO substrate with a spin rate of 2000 rpm for 40 seconds, and then baked at 120° C. in air for 5 minutes to form an electron transporting layer (ETL). The active layer solution comprises the aforementioned organic composition, wherein at least one P-type organic semiconductor material is used as a donor material, and at least one N-type organic semiconductor material is used as an acceptor material (the weight ratio of donor material to acceptor material is 1:1˜2). The concentration of the donor material was 10˜20 mg/mL. In order to completely dissolve the active layer material, the active layer solution needs to be stirred on a hot plate at 100° C. for at least 3 hours. After completely dissolving the active layer material, the active layer solution is filtered with PTFE filter membrane (pore size 0.45˜1.2 μm) and heated for 1 hour. Then, the active layer solution is cooled to the room temperature for spin coating, and the spin rate was used to control the film thickness in the range of 100-800 nm. Finally, the thin film formed by the coated active layer is annealed at 100° C. for 5 minutes, and then transferred to a thermal evaporation machine. A thin layer (8 nm) of MoO₃ is deposited as a hole transporting layer (HTL) under a vacuum of 3×10⁻⁶ Torr. In this experiment, a Keithley™ 2400 source meter was used to record the dark current density (J_{dark}, at a bias of 0˜−8 V) in the absence of light. External quantum efficiency system was used to measure external quantum efficiency (EQE) with a range of 300-1100 nm (bias voltage 0˜−8 V), and silicon (300-1100 nm) is used for light source calibration.

It should be noted here that, in practical applications, the first electrode preferably has good light transmittance. The first electrode is usually made of the transparent conductive material, preferably selected from one of the following conductive material groups: indium oxide, tin oxide, fluorine-doped tin oxide (FTO) derivative, or composite metal oxides such as indium tin oxide (ITO) and indium zinc oxide (IZO). The material of the second electrode is a conductive metal, preferably silver or aluminum, more preferably silver. Suitable and preferred materials for electron transporting layer include, but are not limited to, metal oxides such as ZnOₓ, aluminum doped ZnO (AZO), TiOₓ or nanoparticles thereof, salts (such as LiF, NaF, CsF, Cs₂CO₃), amines (such as primary amines, secondary or tertiary amines), conjugated polymer electrolytes (such as polyethyleneimine), conjugated polymers (such as poly[3-(6-trimethylammoniumhexyl)thiophene], poly(9,9)-bis(2-ethylhexyl-fluorene)-b-poly[3-(6-trimethylammoniumhexyl)thiophene] or poly[(9,9-bis(3′-(N,N-dimethylamino)) propyl)-2,7-fluorene)-alt-2,7-(9,9-dioctylfluorene)], and organic compounds such as tris(8-quinolinyl)-aluminum (III) (Alq₃), 4,7-diphenyl-1,10-phenanthroline, or a combination of one or more of the foregoing. Suitable and preferred materials for hole transporting layer include, but are not limited to metal oxides such as ZTO (Zinc Tin Oxide), MoOₓ, WOₓ, NiOₓ, SnOₓ or nanoparticles thereof, metal-containing salts, such as copper sulfide, copper thiocyanate, copper iodide, copper indium sulfide, lead sulfide, cobalt acetate, tungsten disulfide, etc., conjugated polymer electrolytes such as PEDOT:PSS, polymeric acids such as polyacrylates, conjugated polymers such as polytriarylamine (PTAA), insulating polymers such as Nafion films, polyethyleneimine or polystyrene sulfonates, organic compounds such as N,N′-diphenyl-N,N′-bis(1-naphthyl) (1,1′-biphenyl)-4,4′-diamine (NPB), N,N′-diphenyl-N,N′-(3-methylbenzene base)-1,1′-biphenyl-4,4′-diamine (TPD), or a combination of one or more of the above.

Please refer to Table 2. Table 2 shows the dark current density test results of the organic optoelectronic devices of Example 1, Example 3, Example 6 and Comparative Example 1 in the annealing test.

TABLE 2
the dark current density test results of the organic
optoelectronic devices of Example 1, Example 3, Example
6 and Comparative Example 1 in the annealing test.

| PBDB-T:N-type | J_{dark} (A/cm²) @ −4 V | | |
|---|---|---|---|
| (1:1) | Initial | 1 hr | 2 hr |
| Example 1 | 1.96E−07 | 3.82E−08 | 2.11E−08 |
| Example 3 | 4.15E−08 | 2.17E−08 | 2.25E−08 |
| Example 6 | 4.50E−08 | 3.28E−08 | 2.21E−08 |
| Comparative | 1.20E−08 | 3.64E−08 | 3.33E−08 |
| example 1 | | | |

In the preparation of organic optoelectronic devices, organic material Example 1, Example 3, Example 6 and Comparative Example 1 are used as N-type organic semiconductor materials, and organic optoelectronic devices Example 1, Example 3, Example 6 and Comparative Example 1 are prepared with PBDB-T as a P-type organic semiconductor material. The organic optoelectronic devices are subjected to device testing to investigate its initial performance of dark current density and the device performance after annealing at 160° C. for 1 hour and 2 hours. As shown in Table 2, in terms of initial device performance, Example 1, Example 3 and Example 6 have higher dark current density at −4V. After annealing at 160° C., in the trend of dark current density at −4V, Example 1, Example 3 and Example 6 all showed a decreasing trend, while Comparative Example 1 showed an increasing trend. In the application of organic photodetectors, a lower dark current density is highly desirable, as it improves the signal-to-noise ratio and enhances detectivity. As shown in Table 2, the dark current density of Comparative Example 1 increases due to annealing. In particular, after two hours of annealing, the dark current densities of Example 1, Example 3, and Example 6 are 2.11×10⁻⁸, 2.25×10⁻⁸ and 2.21×10⁻⁸ A/cm², while Comparative Example 1 is 3.33×10⁻⁸ A/cm². The dark current density of Comparative Example 1 changes from the originally lowest to the highest. It can be seen that, compared with Comparative Example 1, the organic optoelectronic devices of Example 1, Example 3 and Example 6 of the present invention have better device thermal stability.

Furthermore, the performance test of the organic photodetectors was carried out using the organic optoelectronic devices Example 1 and the Comparative Example 1.

Please refer to Table 3. Table 3 shows the performance test of the organic optoelectronic devices Example 1 and Comparative Example 1.

TABLE 3
the performance test of the organic optoelectronic
devices Example 1 and Comparative Example 1.

| | | | | | | | Retention D* | | |
|---|---|---|---|---|---|---|---|---|---|
| | J_{dark} | | | EQE at 940 | | | at 940 nm | | |
| PBDB-T:Acceptor | (A/cm²) @ −4 V | | | nm (%) @ −4 V | | | (%) @ −4 V | | |
| (1:1) | Initial | 1 hr | 2 hr | Initial | 1 hr | 2 hr | Initial | 1 hr | 2 hr |
| Example 1 | 1.96E−07 | 3.82E−08 | 2.11E−08 | 54.1 | 56.3 | 51.8 | 100% | 236% | 292% |
| Comparative | 1.20E−08 | 3.64E−08 | 3.33E−08 | 48.1 | 29.2 | 29.0 | 100% | 35% | 36% |
| example 1 | | | | | | | | | |

In the preparation of organic optoelectronic devices, organic material Example 1 and Comparative Example 1 are used as N-type organic semiconductor materials, and organic optoelectronic devices Example 1 and Comparative Example 1 are prepared with PBDB-T as a P-type organic semiconductor material. As shown in Table 3, in terms of initial device performance, Example 1 has a higher external quantum efficiency (EQE) performance and a higher dark current density. After annealing at 160° C., in the trend of dark current density at −4V, Example 1 gradually decreases from 1.96×10⁻⁷ A/cm² to 2.11×10⁻⁸ A/cm², while the dark current density of Comparative Example 1 is gradually increased from 1.20×10⁻⁸ A/cm² to 3.33×10⁻⁸ A/cm². As for the trend of EQE, Example 1 slightly decreased from 54.1% to 51.8%, while Comparative Example 1 significantly decreased from 48.1% to 29.0%. After calculating the detectivity of the organic optoelectronic devices, we set the initial value as 100%. After annealing at 160° C., Example 1 increased to 292%, while Comparative Example 1 decreased to only 36%. It can be seen from this that the organic optoelectronic devices of the present invention have good device thermal stability.

Based on the above experimental results, the organic material, organic composition and organic optoelectronic devices using the organic material containing the formula I of the present invention as organic photodetectors have the following characteristics: (1) the device manufacturing process does not require the use of toxic halogenated solvents; (2) it has low dark current density and good EQE and detection performance in the near-infrared light band; and (3) the materials and devices have good thermal stability.

With the detailed description of the above embodiments, it is hoped that the features and spirit of the present invention can be more clearly described, and the scoped of the present invention is not limited by the embodiments disclosed above. On the contrary, the intention is to cover various changes and equivalent arrangements within the scope of the patents to be applied for in the present invention.

What is claimed is:

1. An organic material comprises a structure such as Formula I: 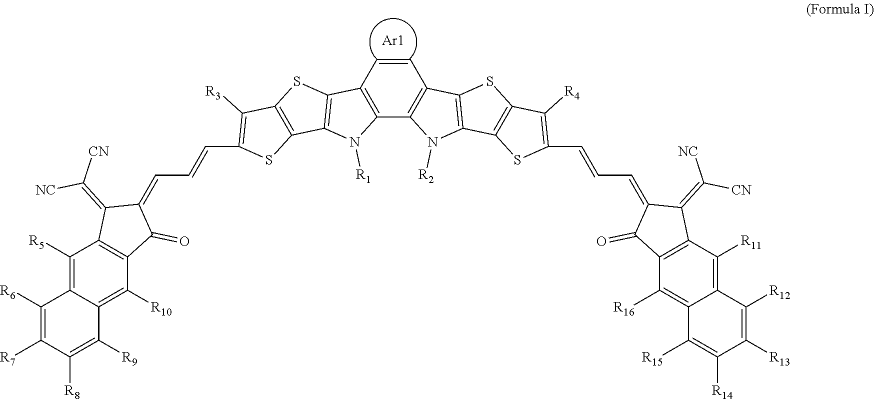
wherein Ar1 is a monocyclic ring or polycyclic ring comprising at least one five-membered heterocycle or six-membered heterocycle with one or more heteroatoms, and the heteroatoms are independently selected from at least one of S, N, O, and Se;
R₁, R₂, R₃, and R₄ are independently selected from the following groups and their derivatives: C1-C30 alkyl, C1-C30 silyl, C1-C30 alkoxy, C1-C30 alkylthio, C1-C30 haloalkyl, C2-C30 ester, C1-C30 alkylaryl, C1-C30 alkyl heteroaryl, C1-C30 silyl aryl, C1-C30 silyl heteroaryl, C1-C30 alkoxyaryl, C1-C30 alkoxy heteroaryl, C1-C30 alkylthioaryl, C1-C30 alkylthio heteroaryl, C1-C30 haloalkyl aryl, C1-C30 haloalkyl heteroaryl, C2-C30 esteryl aryl, and C2-C30 esteryl heteroaryl; and
R₅, R₆, R₇, R₈, R₉, R₁₀, R₁₁, R₁₂, R₁₃, R₁₄, R₁₅ and R₁₆ are independently selected from the following groups and their derivatives: C1-C30 alkyl, C1-C30 silyl, C1-C30 alkoxy, C1-C30 alkylthio, C1-C30 haloalkyl, halogen, hydrogen, deuterium, tritium, and cyano group.

2. The organic material of claim 1, wherein an optical band gap of the organic material is <1.25 eV.

3. The organic material of claim 1, wherein Ar1 is selected from the following structures: 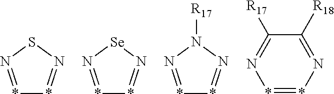
wherein each structure is connected by *, and R₁₇ and R₁₈ are independent single groups or connected to each other by covalent bonds to form a combined group.

4. The organic material of claim 3, wherein R₁₇ and R₁₈ are selected from the following groups and their derivatives: halogen, hydrogen, cyano, C1-C30 alkyl, C2-C30 alkenyl, C1-C30 alkoxy, C1-C30 alkylthio, C1-C30 halogenated alkyl, C2-C30 ester, C1-C30 alkylaryl, C1-C30 alkyl heteroaryl, C1-C30 silyl aryl, C1-C30 silyl heteroaryl, C1-C30 alkoxyaryl, C1-C30 alkoxy heteroaryl, C1-C30 alkylthioaryl, C1-C30 alkylthioheteroaryl, C1-C30 halogenated alkylaryl, C1-C30 halogenated alkyl heteroaryl, C2-C30 esteryl aryl and C2-C30 esteryl heteroaryl.

5. The organic material of claim 1, wherein R₁ and R₂ are further independently selected from the following groups and their derivatives: C1-C30 alkyl, C1-C30 alkylaryl and C1-C30 alkyl heteroaryl; and R₃ and R₄ are further independently selected from the following groups and their derivatives: C1-C30 alkyl, C1-C30 silyl, C1-C30 alkoxy, C1-C30 alkylthio, C1-C30 alkylaryl, C1-C30 alkyl heteroaryl, C1-C30 alkoxyaryl, C1-C30 alkoxy heteroaryl, C1-C30 alkylthioaryl, C1-C30 alkylthio heteroaryl, C1-C30 haloalkyl aryl and C1-C30 haloalkyl heteroaryl.

6. The organic material of claim 1, wherein R₅, R₆, R₇, R₈, R₉, R₁₀, R₁₁, R₁₂, R₁₃, R₁₄, R₁₅ and R₁₆ are independently selected from the following groups: halogen, hydrogen, deuterium, and cyano group, and R₅, R₆, R₇, R₈, R₉, R₁₀, R₁₁, R₁₂, R₁₃, R₁₄, R₁₅ and R₁₆ are not hydrogen atoms at same time.

7. The organic material of claim 1, wherein R₅, R₁₀, R₁₁ and R₁₆ are further selected from hydrogen; and R₆, R₇, R₈, R₉, R₁₂, R₁₃, R₁₄ and R₁₅ are further independently selected from the following groups and their derivatives: halogen, hydrogen, C1-C5 haloalkyl, C1-C5 alkoxy, and cyano group.

8. An organic composition comprising at least one P-type organic semiconductor material and at least one N-type organic semiconductor material, wherein the P-type organic semiconductor material comprises at least one of organic conjugated polymer or organic conjugated small molecule; and the N-type organic semiconductor material comprises at least one organic material of claim 1.

9. An organic optoelectronic device comprising:
a first electrode;
an active layer which at least comprises the organic material of claim 1; and
a second electrode, wherein the active layer is disposed between the first electrode and the second electrode, and at least one of the first electrode and the second electrode is a transparent or semi-transparent electrode.

10. An organic optoelectronic device comprising:
a first electrode;
an active layer which at least comprises the organic composition of claim 8; and
a second electrode, wherein the active layer is disposed between the first electrode and the second electrode, and at least one of the first electrode and the second electrode is a transparent or semi-transparent electrode.
